(19) United States
(12) Patent Application Publication
(10) Pub. No.: US 2019/0093128 A1
(43) Pub. Date: Mar. 28, 2019

(54) METHODS FOR GENOME EDITING IN ZYGOTES

(71) Applicant: The Regents of the University of California, Oakland, CA (US)

(72) Inventors: Sean Chen, Berkeley, CA (US); Andrew J. Modzelewski, Berkeley, CA (US); Lin He, Berkeley, CA (US); Angus Lee, Berkeley, CA (US)

(21) Appl. No.: 16/084,158

(22) PCT Filed: Mar. 30, 2017
(86) PCT No.: PCT/US17/25039

§ 371 Date: Sep. 11, 2018

Related U.S. Application Data
(60) Provisional application No. 62/316,289, filed on Mar. 31, 2016.

(51) Int. Cl. C12N 15/873; C12N 9/22; C12N 15/11

(52) U.S. Cl. CPC C12N 15/873; C12N 2310/20; C12N 15/11; C12N 9/22

(57) ABSTRACT

The present disclosure provides methods of modifying the genome of a mammalian zygote. The present disclosure provides methods of modulating transcription in a mammalian zygote. The present disclosure provides methods of labeling a target nucleic acid in the genome of a mammalian zygote. The present disclosure provides methods of delivering a ribonucleoprotein complex into a mammalian zygote. The present disclosure provides methods of delivering a polypeptide or a nucleic acid into a mammalian zygote.

### CROSS-REFERENCE

This application claims the benefit of U.S. Provisional Patent Application No. 62/316,289, filed Mar. 31, 2016, which application is incorporated herein by reference in its entirety.

### STATEMENT REGARDING FEDERALLY SPONSORED RESEARCH

This invention was made with government support under Grant No. CA192636 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “BERK-324PRV_SeqList_ST25.txt” created on Mar. 6, 2016 and having a size of 7,914 KB. The contents of the text file are incorporated by reference herein in their entirety.

### INTRODUCTION

Easily accessible and efficient methodologies to edit the genomes of organisms are an immense resource to the biological and biomedical research community. Traditionally, engineering of the mammalian genome is achieved by homologous recombination (HR)-mediated sequence substitution in embryonic stem cells (ESCs), a time consuming process that occurs at low frequency. Taking genetically engineering in mice for example, after extensive screening for ESC colonies with the desired genetic modifications, ESCs are microinjected into mouse blastocysts to generate chimeras capable of germline transmission. Such chimera mice are then crossed to wild-type mice to generate heterozygous offspring (F1), which are then intercrossed to yield homozygous mutant mice (F2) that can be subjected to phenotypic analyses. Despite the wide use of this technology to generate transgenic mice, the low efficiency of HR in ESCs, the laborious process of screening, the technical difficulty of microinjection, and the nature of the mouse life cycle make this approach a lengthy and costly process.

### SUMMARY

The present disclosure provides methods of modifying the genome of a mammalian zygote. The present disclosure provides methods of modulating transcription in a mammalian zygote. The present disclosure provides methods of labeling a target nucleic acid in the genome of a mammalian zygote. The present disclosure provides methods of delivering a ribonucleoprotein complex into a mammalian zygote. The present disclosure provides methods of delivering a polypeptide or a nucleic acid into a mammalian zygote.

The present disclosure provides a method of modifying genomic DNA of a mammalian zygote, the method comprising introducing into the zygote a ribonucleoprotein (RNP) comprising a class 2 CRISPR/Cas endonuclease complexed with a corresponding CRISPR/Cas guide RNA that hybridizes to a target sequence within the genomic DNA of the zygote, wherein said introducing is by electroporation of an electroporation composition comprising the RNP and the zygote, and wherein said introducing results in modification of the genomic DNA. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. In some cases, the Cas9 guide RNA is a single guide RNA (sgRNA). In some cases, the RNP comprises two or more CRISPR/Cas guide RNAs. In some cases, the class 2 CRISPR/Cas endonuclease is a type V or type VI CRISPR/Cas endonuclease. In some cases, the class 2 CRISPR/Cas polypeptide is a Cpf1 polypeptide, a C2c1 polypeptide, a C2c3 polypeptide, or a C2c2 polypeptide. In some cases, modification of the genomic DNA is homozygous modification. In some cases, modification of the genomic DNA is heterozygous modification. In some cases, the modification comprises deletion of genomic DNA, insertion of a nucleic acid into the genomic DNA, or both deletion of genomic DNA and insertion of a nucleic acid into the genomic DNA. In some cases, the modification comprises inversion of genomic DNA. In some cases, the modification comprises insertion of a nucleic acid into genomic DNA. In some cases, the modification comprises replacement of genomic DNA. In some cases, the method comprises introducing into the zygote a donor DNA. In some cases, the zygote is a rodent zygote. In some cases, the zygote is a mouse zygote or a rat zygote. In some cases, the zygote is a rabbit zygote, a cat zygote, a dog zygote, or a horse zygote. In some cases, the zygote is an ungulate zygote. In some cases, the zygote is a human zygote. In some cases, the zygote is a non-human primate zygote. In some cases, the zygote is a non-human mammalian zygote. In some cases, the electroporation comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation composition”); and b) electroporating the zygote/RNP complex composition with: i) one or more pulses of 1 millisecond to 6 milliseconds in duration; ii) one or more pulses of 1 millisecond to 6 milliseconds in duration, where each pulse is 30 V; iii) a single pulse at 30 V, where the pulse is a 3-millisecond (msec) pulse; or iv) 6 pulses of 30 V each, where each pulse is 3 milliseconds in duration. In some cases, the RNP is present in the electroporation composition at a concentration of from 5 μM to 16 μM. In some cases, the RNP is present in the electroporation composition at a concentration of 8 μM. In some cases, at least 50%, at least 60%, at least 70%, at least 80%, at least 85%, at least 90%, at least 95%, or at least 98%, of the zygotes are viable after electroporation with the RNP. In some cases, the genomic modification occurs via homology-directed repair (HDR) or non-homologous end joining (NHEJ). In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

The present disclosure provides a method of modulating transcription in a mammalian zygote, the method comprising introducing into the zygote a ribonucleoprotein (RNP) comprising an enzymatically inactive CRISPR/Cas9 polypeptide complexed with a CRISPR/Cas guide RNA that hybridizes to a target sequence within the genomic DNA of the zygote, wherein said introducing is by electroporation of an electroporation composition comprising the RNP and the zygote, and wherein said introducing results in modulation of transcription of a gene comprising the target sequence. In some cases, the zygote is a rodent zygote. In some cases, the zygote is a mouse zygote or a rat zygote. In some cases, the zygote is a rabbit zygote, a cat zygote, a dog zygote, or a horse zygote. In some cases, the zygote is an ungulate zygote. In some cases, the zygote is a human zygote. In some cases, the zygote is a non-human primate zygote. In some cases, wherein the electroporation comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation composition”); and b) electroporating the zygote/RNP complex composition with: i) one or more pulses of 1 millisecond to 6 milliseconds in duration; ii) one or more pulses of 1 millisecond to 6 milliseconds in duration, where each pulse is 30 V; iii) a single pulse at 30 V, where the pulse is a 3-millisecond (msec) pulse; or iv) 6 pulses of 30 V each, where each pulse is 3 milliseconds in duration. In some cases, the zygote is a non-human primate zygote. In some cases, wherein the electroporation comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation composition”); and b) electroporating the zygote/RNP complex composition with one or more pulses of 1 millisecond to 6 milliseconds in duration. In some cases, wherein the electroporation comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation composition”); and b) electroporating the zygote/RNP complex composition with one or more 30 V pulses of 1 millisecond to 6 milliseconds in duration. In some cases, electroporating the zygote/RNP complex composition comprises electroporating with one or more pulses (e.g., applying one or more pulses to the zygote(s)/RNP complex composition). In some cases, a method of the present disclosure comprises electroporating a zygote/RNP complex composition with a single pulse. In some cases, a method of the present disclosure comprises electroporating a zygote/RNP complex composition with a single pulse of 1 millisecond to 5 milliseconds in duration. In some cases, a method of the present disclosure comprises electroporating a zygote/RNP complex composition with a single 30 V pulse. In some cases, a method of the present disclosure comprises electroporating a zygote/RNP complex composition with a single 30 V pulse of 1 millisecond to 5 milliseconds in duration. In some cases, a method of the present disclosure comprises electroporating a zygote/RNP complex composition with 2 pulses. In some cases, a method of the present disclosure comprises electroporating a zygote/RNP complex composition with 2 pulses, each pulse 1 millisecond to 5 milliseconds in duration. In some cases, a method of the present disclosure comprises electroporating a zygote/RNP complex composition with 2 pulses at 30 V each. In some cases, a method of the present disclosure comprises electroporating a zygote/RNP complex composition with 2 pulses at 30 V each, where each pulse is 1 millisecond to 5 milliseconds in duration. In some cases, a method of the present disclosure comprises electroporating a zygote/RNP complex composition with 3 pulses. In some cases, a method of the present disclosure comprises electroporating a zygote/RNP complex composition with 3 pulses, each pulse 1 millisecond to 5 milliseconds in duration. In some cases, a method of the present disclosure comprises electroporating a zygote/RNP complex composition with 3 pulses at 30 V each. In some cases, a method of the present disclosure comprises electroporating a zygote/RNP complex composition with 3 pulses at 30 V each, where each pulse is 1 millisecond to 5 milliseconds in duration. In some cases, a method of the present disclosure comprises electroporating a zygote/RNP complex composition with 4 pulses. In some cases, a method of the present disclosure comprises electroporating a zygote/RNP complex composition with 4 pulses, each pulse 1 millisecond to 5 milliseconds in duration. In some cases, a method of the present disclosure comprises electroporating a zygote/RNP complex composition with 4 pulses at 30 V each. In some cases, a method of the present disclosure comprises electroporating a zygote/RNP complex composition with 4 pulses at 30 V each, where each pulse is 1 millisecond to 5 milliseconds in duration. In some cases, a method of the present disclosure comprises electroporating a zygote/RNP complex composition with 5 pulses. In some cases, a method of the present disclosure comprises electroporating a zygote/RNP complex composition with 5 pulses, each pulse 1 millisecond to 5 milliseconds in duration. In some cases, a method of the present disclosure comprises electroporating a zygote/RNP complex composition with 5 pulses at 30 V each. In some cases, a method of the present disclosure comprises electroporating a zygote/RNP complex composition with 5 pulses at 30 V each, where each pulse is 1 millisecond to 5 milliseconds in duration. In some cases, a method of the present disclosure comprises electroporating a zygote/RNP complex composition with 6 pulses. In some cases, a method of the present disclosure comprises electroporating a zygote/RNP complex composition with 6 pulses, each pulse 1 millisecond to 5 milliseconds in duration. In some cases, a method of the present disclosure comprises electroporating a zygote/RNP complex composition with 6 pulses at 30 V each. In some cases, a method of the present disclosure comprises electroporating a zygote/RNP complex composition with 6 pulses at 30 V each, where each pulse is 1 millisecond to 5 milliseconds in duration. In some cases, electroporation comprises electroporating with one or more pulses at 30 V (i.e., 30 V each pulse), where the one or more pulses is a 3-millisecond (msec) pulse. In some cases, the one or more pulses is 6 pulses. In some cases, a method of the present disclosure comprises electroporating a zygote/RNP complex composition with 7 pulses. In some cases, a method of the present disclosure comprises electroporating a zygote/RNP complex composition with 7 pulses, each pulse 1 millisecond to 5 milliseconds in duration. In some cases, a method of the present disclosure comprises electroporating a zygote/RNP complex composition with 7 pulses at 30 V each. In some cases, a method of the present disclosure comprises electroporating a zygote/RNP complex composition with 7 pulses at 30 V each, where each pulse is 1 millisecond to 5 milliseconds in duration. In some cases, a method of the present disclosure comprises electroporating a zygote/RNP complex composition with 8 pulses. In some cases, a method of the present disclosure comprises electroporating a zygote/RNP complex composition with 8 pulses, each pulse 1 millisecond to 5 milliseconds in duration. In some cases, a method of the present disclosure comprises electroporating a zygote/RNP complex composition with 8 pulses at 30 V each. In some cases, a method of the present disclosure comprises electroporating a zygote/RNP complex composition with 8 pulses at 30 V each, where each pulse is 1 millisecond to 5 milliseconds in duration. In some cases, a method of the present disclosure comprises electroporating a zygote/RNP complex composition with 9 pulses. In some cases, a method of the present disclosure comprises electroporating a zygote/RNP complex composition with 9 pulses, each pulse 1 millisecond to 5 milliseconds in duration. In some cases, a method of the present disclosure comprises electroporating a zygote/RNP complex composition with 9 pulses at 30 V each. In some cases, a method of the present disclosure comprises electroporating a zygote/RNP complex composition with 9 pulses at 30 V each, where each pulse is 1 millisecond to 5 milliseconds in duration. In some cases, a method of the present disclosure comprises electroporating a zygote/RNP complex composition with 10 pulses. In some cases, a method of the present disclosure comprises electroporating a zygote/RNP complex composition with 10 pulses, each pulse 1 millisecond to 5 milliseconds in duration. In some cases, a method of the present disclosure comprises electroporating a zygote/RNP complex composition with 10 pulses at 30 V each. In some cases, a method of the present disclosure comprises electroporating a zygote/RNP complex composition with 10 pulses at 30 V each, where each pulse is 1 millisecond to 5 milliseconds in duration.

The present disclosure provides a method of labelling a genomic DNA in a mammalian zygote, the method comprising introducing into the zygote a ribonucleoprotein (RNP) comprising an enzymatically inactive CRISPR/Cas9 polypeptide complexed with a CRISPR/Cas guide RNA that hybridizes to a target sequence within the genomic DNA of the zygote, wherein said introducing is by electroporation of an electroporation composition comprising the RNP and the zygote, and wherein said introducing results in labelling of the genomic DNA. In some cases, the zygote is a rodent zygote. In some cases, the zygote is a mouse zygote or a rat zygote. In some cases, the zygote is a rabbit zygote, a cat zygote, a dog zygote, or a horse zygote. In some cases, the zygote is an ungulate zygote. In some cases, the zygote is a human zygote. In some cases, the zygote is a non-human primate zygote. In some cases, the zygote is a non-human mammalian zygote. In some cases, the electroporation comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation composition”); and b) electroporating the zygote/RNP complex composition with: i) one or more pulses of 1 millisecond to 6 milliseconds in duration; ii) one or more pulses of 1 millisecond to 6 milliseconds in duration, where each pulse is 30 V; iii) a single pulse at 30 V, where the pulse is a 3-millisecond (msec) pulse; or iv) 6 pulses of 30 V each, where each pulse is 3 milliseconds in duration.

The present disclosure provides a method of delivering a ribonucleoprotein (RNP) complex into a mammalian zygote, the method comprising electroporating a composition comprising the mammalian zygote and the RNP complex, thereby delivering the RNP complex into the zygote. In some cases, the RNP complex comprises an siRNA, an shRNA, a modified RNA, or a DNA nucleic acid.

The present disclosure provides a method of delivering a nucleic acid into a mammalian zygote, the method comprising electroporating a composition comprising the mammalian zygote and the nucleic acid, thereby delivering the nucleic acid into the zygote.

The present disclosure provides a method of delivering a polypeptide into a mammalian zygote, the method comprising electroporating a composition comprising the mammalian zygote and the polypeptide, thereby delivering the polypeptide into the zygote.

In any of the methods described above or elsewhere herein, in some cases, the zygote is a rodent zygote. In any of the methods described above or elsewhere herein, in some cases, the zygote is a mouse zygote or a rat zygote. In any of the methods described above or elsewhere herein, in some cases, the zygote is a rabbit zygote, a cat zygote, a dog zygote, or a horse zygote. In any of the methods described above or elsewhere herein, in some cases, the zygote is an ungulate zygote. In any of the methods described above or elsewhere herein, in some cases, the zygote is a human zygote. In any of the methods described above or elsewhere herein, in some cases, the zygote is a non-human primate zygote. In any of the methods described above or elsewhere herein, in some cases, the zygote is a non-human mammalian zygote. In any of the methods described above or elsewhere herein, in some cases, the electroporation comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of the RNP complex, the nucleic acid, or the polypeptide, forming an electroporation composition (an “electroporation composition”); and b) electroporating the electroporation composition with i) one or more pulses of 1 millisecond to 6 milliseconds in duration; ii) one or more pulses of 1 millisecond to 6 milliseconds in duration, where each pulse is 30 V; iii) a single pulse at 30 V, where the pulse is a 3-millisecond (msec) pulse; or iv) 6 pulses of 30 V each, where each pulse is 3 milliseconds in duration.

In any of the methods described above or elsewhere herein, in some cases, at least 50%, at least 60%, at least 70%, at least 80%, at least 85%, at least 90%, at least 95%, or at least 98%, of the zygotes are viable after electroporation.

### BRIEF DESCRIPTION OF THE DRAWINGS

FIG. 1A-1H depict generation of NHEJ-mediated indel mutations using CRISPR-EZ.

FIG. 2A-2F depict generation of HDR-mediated point mutations using CRISPR-EZ.

FIG. 3 provides Table 1.

FIG. 4 provides Table 2.

FIGS. 5A and 5B provide Table 3 (FIG. 5A) and Table 4 (FIG. 5B).

FIG. 6 provides the amino acid sequence of a Staphylococcus aureus Cas9 polypeptide.

FIG. 7 provides the amino acid sequence of a Streptococcus pyogenes Cas9 polypeptide.

FIG. 8 provides the amino acid sequence of a high-fidelity (HF) Cas9 polypeptide.

FIG. 9A-9C depict deletion of a retrotransposon upstream of Cdk2ap1.

FIG. 10A-10D depict optimization of CRISPR-EZ efficiency, throughput, and robustness to achieve enhanced genome editing efficiency and survival.

FIG. 11 provides a table showing zygotes treated with CRISPR-EZ and transferred to pseudopregnant recipient females that gave birth to edited mice.

FIG. 12 provides a table showing zygotes treated with CRISPR-EZ and developed into the morula stage.

### DEFINITIONS

By “site-directed modifying polypeptide” or “site-directed DNA modifying polypeptide” or “site-directed target nucleic acid modifying polypeptide” or “RNA-binding site-directed polypeptide” or “RNA-binding site-directed modifying polypeptide” or “site-directed polypeptide” it is meant a polypeptide that binds a guide RNA and is targeted to a specific DNA sequence by the guide RNA. A site-directed modifying polypeptide can be class 2 CRISPR/Cas protein (e.g., a type II CRISPR/Cas protein, a type V CRISPR/Cas protein, a type VI CRISPR/Cas protein). An example of a type II CRISPR/Cas protein is a Cas9 protein (“Cas9 polypeptide”). Examples of type V CRISPR/Cas proteins are Cpf1, C2c1, and C2c3. An example of a type II CRISPR/Cas protein is a C2c2 protein. Class 2 CRISPR/Cas proteins (e.g., Cas9, Cpf1, C2c1, C2c2, and C2c3) as described herein are targeted to a specific DNA sequence by the RNA (a guide RNA) to which it is bound. The guide RNA comprises a sequence that is complementary to a target sequence within the target DNA, thus targeting the bound CRISPR/Cas protein to a specific location within the target DNA (the target sequence). For example, a Cpf1 polypeptide as described herein is targeted to a specific DNA sequence by the RNA (a guide RNA) to which it is bound. The guide RNA comprises a sequence that is complementary to a target sequence within the target DNA, thus targeting the bound Cpf1 protein to a specific location within the target DNA (the target sequence).

“Heterologous,” as used herein, means a nucleotide or polypeptide sequence that is not found in the native nucleic acid or protein, respectively.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes glycoproteins, lipoproteins, phosphoproteins, immunologically tagged proteins, fusion proteins, and the like.

The term “naturally-occurring” as used herein as applied to a nucleic acid, a cell, or an organism, refers to a nucleic acid, cell, or organism that is found in nature. For example, a polypeptide or polynucleotide sequence that is present in an organism (including viruses) that can be isolated from a source in nature and which has not been intentionally modified by a human in the laboratory is naturally occurring.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

As used herein, the term “exogenous nucleic acid” refers to a nucleic acid that is not normally or naturally found in and/or produced by a given cell in nature. As used herein, the term “endogenous nucleic acid” refers to a nucleic acid that is normally found in and/or produced by a given cell in nature. An “endogenous nucleic acid” is also referred to as a “native nucleic acid” or a nucleic acid that is “native” to a given cell.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such can be done to replace a codon with a redundant codon encoding the same or a conservative amino acid, while typically introducing or removing a sequence recognition site. It can also be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant” polypeptide refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA.

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The term “zygote” is well understood in the art, and refers to a diploid cell resulting from the fusion of two haploid gametes.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a CRISPR/Cas endonuclease” includes a plurality of such endonucleases and reference to “the ribonucleoprotein” includes reference to one or more ribonucleoproteins and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of modifying the genome of a mammalian zygote. The present disclosure provides methods of modulating transcription in a mammalian zygote. The present disclosure provides methods of labeling a target nucleic acid in the genome of a mammalian zygote. The present disclosure provides methods of delivering a ribonucleoprotein complex into a mammalian zygote.

Methods of Delivering a Ribonucleoprotein Complex into a Zygote

The present disclosure provides methods of delivering a ribonucleoprotein (RNP) complex into a mammalian zygote.

In some cases, the RNP complex comprises an siRNA, a microRNA, an antisense RNA, an shRNA, a modified RNA, an antagomir RNA, or a DNA nucleic acid. In some cases, the RNP complex comprises an RNAi agent (e.g., an siRNA, an shRNA, etc.).

In some cases, the RNP complex comprises an antisense agent. An antisense agent may be antisense oligonucleotides (ODN), e.g., synthetic ODN having chemical modifications from native nucleic acids, or nucleic acid constructs that express such antisense molecules as RNA. The antisense sequence is complementary to the targeted mRNA, and inhibits its translation into protein. One or a combination of antisense molecules may be used, where a combination may comprise multiple different sequences.

Antisense molecules may be produced by expression of all or a part of a target nucleotide sequence in an appropriate vector, where the transcriptional initiation is oriented such that an antisense strand is produced as an RNA molecule. Alternatively, the antisense molecule may be a synthetic oligonucleotide. Antisense oligonucleotides will generally be at least about 7, e.g., at least about 12, at least about 20 nucleotides in length, or not more than about 25, e.g., not more than about 23-22 nucleotides in length, where the length is governed by efficiency of inhibition, specificity, including absence of cross-reactivity, and the like.

Antisense oligonucleotides may be chemically synthesized by methods known in the art. In some cases, oligonucleotides are chemically modified from the native phosphodiester structure, in order to increase their intracellular stability and binding affinity. A number of modifications that alter the chemistry of the backbone, sugars or heterocyclic bases have been described in the literature, any of which may be included in the antisense agent. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

In some cases, the RNP complex comprises an RNAi agent. By RNAi agent is meant an agent that modulates expression of a gene by an RNA interference mechanism.

The RNAi agents are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. In certain embodiments, the oligoribonucleotide is less than 100, 95, 90, 85, 80, 75, 70, 65, 60, 55, 50, 45 or 40 nt in length. In certain embodiments, the oligoribonucleotide is less than 100 nt in length. In other embodiments, the oligoribonucleotide is less than 95 nt in length. In another embodiment, the oligoribonucleotide is less than 90 nt in length. In another embodiment, the oligoribonucleotide is less than 85 nt in length. In some embodiments, the oligoribonucleotide is less than 80 nt in length. In other embodiments, the oligoribonucleotide is less than 75 nt in length. In other embodiments, the oligoribonucleotide is less than 70 nt in length. In other embodiments, the oligoribonucleotide is less than 65 nt in length. In yet other embodiments, the oligoribonucleotide is less than 60 nt in length. In other embodiments, the oligoribonucleotide is less than 55 nt in length. In certain embodiments, the oligoribonucleotide is less than 50 nt in length. In other embodiments, the oligoribonucleotide is less than 45 nt in length. In yet other embodiments, the oligoribonucleotide is less than 40 nt in length. In specific embodiments, the oligoribonucleotide is 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41 or 40 nt in length.

Where the RNA agent is a duplex structure of two distinct ribonucleic acids hybridized to each other, e.g., an siRNA, the length of the duplex structure typically ranges from about 15 to 30 bp, e.g., from about 15 to 29 bp, where lengths between about 20 and 29 bps, e.g., 21 bp, 22 bp, can be used. In certain cases, the RNA agent is 30, 29, 28, 27, 26, 25, 24, 23, 22, 21, 20, 19, 18, 17, 16, 15, 14, 13, 12, 11 or 10 bp in length.

In some cases, the RNP complex comprises a DNA-binding polypeptide. In some cases, the RNP complex comprises a TALE nuclease (a “TALEN”), a zinc-finger endonuclease, or an RNA-guided endonuclease. In some cases, the RNA-guided endonuclease is a CRISPR/Cas endonuclease, as described below.

In some cases, the RNP complex comprises: i) a CRISPR/Cas endonuclease; and ii) only one guide RNA. In some cases, the RNP complex comprises: i) a CRISPR/Cas endonuclease; and ii) two guide RNAs. In some cases, the RNP complex comprises: i) a CRISPR/Cas endonuclease; and ii) more than two guide RNAs. In some cases, the guide RNA is a dual-guide RNA (e.g., a dual-molecule guide RNA). In some cases, the guide RNA is a single-guide RNA (e.g., a single-molecule guide RNA).

A method of the present disclosure involves electroporating an RNP complex into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation mixture” or an “electroporation composition”); and b) electroporating the zygote/RNP complex composition with 2 pulses at 30 V, where each pulse is a 3-millisecond (msec) pulse, with a 1 msec interval between the 2 pulses. In some cases, the RNP is present in the electroporation composition at a concentration of from 5 μM to 16 μM. In some cases, the RNP is present in the electroporation composition at a concentration of 8 μM. In some cases, at least 50%, at least 60%, at least 70%, at least 80%, at least 85%, at least 90%, at least 95%, or at least 98%, of the zygotes are viable after electroporation with the RNP. In some cases, from 50% to 95% of the zygotes are viable after electroporation with the RNP. In some cases, from 60% to 95% of the zygotes are viable after electroporation with the RNP. In some cases, from 70% to 95% of the zygotes are viable after electroporation with the RNP. In some cases, from 80% to 95% of the zygotes are viable after electroporation with the RNP. In some cases, 100% of the zygotes are viable after electroporation with the RNP. In some cases, the genomic modification occurs via homology-directed repair (HDR) or non-homologous end joining (NHEJ). In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation mixture” or an “electroporation composition”); and b) electroporating the zygote/RNP complex composition with a single pulse at 30 V, where the single pulse is a 3-msec pulse. In some cases, the RNP is present in the electroporation composition at a concentration of from 5 μM to 16 μM. In some cases, the RNP is present in the electroporation composition at a concentration of 8 μM. In some cases, at least 50%, at least 60%, at least 70%, at least 80%, at least 85%, at least 90%, at least 95%, or at least 98%, of the zygotes are viable after electroporation with the RNP. In some cases, from 50% to 95% of the zygotes are viable after electroporation with the RNP. In some cases, from 60% to 95% of the zygotes are viable after electroporation with the RNP. In some cases, from 70% to 95% of the zygotes are viable after electroporation with the RNP. In some cases, from 80% to 95% of the zygotes are viable after electroporation with the RNP. In some cases, 100% of the zygotes are viable after electroporation with the RNP. In some cases, the genomic modification occurs via homology-directed repair (HDR) or non-homologous end joining (NHEJ). In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

A method of the present disclosure for delivering an RNP complex into a mammalian zygote can be used to deliver an RNP complex into any of a variety of mammalian zygotes, including, e.g., a human zygote or a non-human mammalian zygote. Non-human mammalian zygotes include, but are not limited to, a rodent zygote (e.g., a rat zygote; a mouse zygote); a lagomorph zygote (e.g., a rabbit zygote); a feline zygote, e.g., a cat zygote; a canine zygote, e.g., a dog zygote; an ovine (e.g., sheep) zygote; a caprine (e.g., goat) zygote; an equine (e.g., horse) zygote; an ungulate zygote; a non-human primate zygote; etc.

In some cases, electroporation of the zygote/RNP composition includes electroporating with one or more pulses. In some cases, electroporation of the zygote/RNP composition includes electroporating with multiple pulses. In some cases, electroporation of the zygote/RNP composition includes electroporating with 3 pulses. In some cases, electroporation of the zygote/RNP composition includes electroporating with 4 pulses. In some cases, electroporation of the zygote/RNP composition includes electroporating with 5 pulses. In some cases, electroporation of the zygote/RNP composition includes electroporating with 6 pulses. In some cases, electroporation of the zygote/RNP composition includes electroporating with 7 pulses. In some cases, electroporation of the zygote/RNP composition includes electroporating with 8 pulses. In some cases, electroporation of the zygote/RNP composition includes electroporating with 9 pulses. In some cases, electroporation of the zygote/RNP composition includes electroporating with 10 pulses. In some cases, at least 20%, at least 30%, at least 40%, at least 50%, at least 60%, at least 70%, at least 80%, at least 85%, at least 90%, at least 95%, or at least 98%, of the zygotes are viable after electroporation. In some cases, from 20% to 50% of the zygotes are viable after electroporation. In some cases, from 50% to 95% of the zygotes are viable after electroporation. In some cases, from 60% to 95% of the zygotes are viable after electroporation. In some cases, from 70% to 95% of the zygotes are viable after electroporation. In some cases, from 80% to 95% of the zygotes are viable after electroporation. In some cases, 100% of the zygotes are viable after electroporation.

In some cases, electroporation of the zygote/RNP composition includes electroporating with one or more pulses, where each pulse is a 1-millisecond (msec) pulse. In some cases, electroporation of the zygote/RNP composition includes electroporating with one or more pulses, where each pulse is a 2-millisecond (msec) pulse. In some cases, electroporation of the zygote/RNP composition includes electroporating with one or more pulses, where each pulse is a 3-millisecond (msec) pulse. In some cases, electroporation of the zygote/RNP composition includes electroporating with one or more pulses, where each pulse is a 4-millisecond (msec) pulse. In some cases, electroporation of the zygote/RNP composition includes electroporating with one or more pulses, where each pulse is a 3-millisecond (msec) pulse. In some cases, electroporation of the zygote/RNP composition includes electroporating with one or more pulses, where each pulse is a 5-millisecond (msec) pulse. In some cases, electroporation of the zygote/RNP composition includes electroporating with one or more pulses, where each pulse is a 6-millisecond (msec) pulse. In some cases, electroporation of the zygote/RNP composition includes electroporating with one or more pulses, where each pulse is a 7-millisecond pulse. In some cases, electroporation of the zygote/RNP composition includes electroporating with one or more pulses, where each pulse is an 8-millisecond pulse. In some cases, electroporation of the zygote/RNP composition includes electroporating with one or more pulses, where each pulse is a 9-millisecond pulse. In some cases, electroporation of the zygote/RNP composition includes electroporating with one or more pulses, where each pulse is a 10-millisecond pulse.

In some cases, electroporation of the zygote/RNP composition includes electroporating with multiple pulses at 10 V. In some cases, electroporation of the zygote/RNP composition includes electroporating with multiple pulses at 15 V. In some cases, electroporation of the zygote/RNP composition includes electroporating with multiple pulses at 20 V. In some cases, electroporation of the zygote/RNP composition includes electroporating with multiple pulses at 25 V. In some cases, electroporation of the zygote/RNP composition includes electroporating with multiple pulses at 30 V. In some cases, electroporation of the zygote/RNP composition includes electroporating with multiple pulses at 35 V. In some cases, electroporation of the zygote/RNP composition includes electroporating with multiple pulses at 40 V. In some cases, electroporation of the zygote/RNP composition includes electroporating with multiple pulses at 45 V. In some cases, electroporation of the zygote/RNP composition includes electroporating with multiple pulses at 50 V. In some cases, electroporation of the zygote/RNP composition includes electroporating with multiple pulses at 55 V. In some cases, electroporation of the zygote/RNP composition includes electroporating with multiple pulses at 60 V.

In some cases, electroporation of the zygote/RNP composition includes electroporating with 2 pulses at 30 volts, where each pulse is a 3-millisecond (msec) pulse. In some cases, electroporation of the zygote/RNP composition includes electroporating with 4 pulses at 30 volts, where each pulse is a 3-millisecond (msec) pulse. In some cases, electroporation of the zygote/RNP composition includes electroporating with 6 pulses at 30 volts, where each pulse is a 3-millisecond (msec) pulse. In some cases, electroporation of the zygote/RNP composition includes electroporating with 8 pulses at 30 volts, where each pulse is a 3-millisecond (msec) pulse. In some cases, electroporation of the zygote/RNP composition includes electroporating with 10 pulses at 30 volts, where each pulse is a 3-millisecond (msec) pulse. In some cases, electroporation of the zygote/RNP composition includes electroporating with 12 pulses at 30 volts, where each pulse is a 3-millisecond (msec) pulse.

Methods of Delivering a Polypeptide or a Polynucleotide into a Zygote

The present disclosure provides methods of delivering a nucleic acid into a mammalian zygote. The present disclosure provides methods of delivering a polypeptide into a mammalian zygote.

A polynucleotide to be delivered into a mammalian zygote using a method of the present disclosure can be single-stranded, double-stranded, or multi-stranded. The polynucleotide to be delivered into a mammalian zygote can be DNA or RNA, genomic DNA, cDNA, DNA-RNA hybrids, or a polymer comprising purine and pyrimidine bases or other natural, chemically or biochemically modified, non-natural, or derivatized nucleotide bases.

A polynucleotide to be delivered into a mammalian zygote using a method of the present disclosure can comprise a nucleotide sequence that encodes a polypeptide (e.g., a therapeutic polypeptide; a transcription activator; a transcription repressor; etc.). A polynucleotide to be delivered into a mammalian zygote using a method of the present disclosure can comprise a nucleotide sequence that encodes a functional RNA. A polynucleotide to be delivered into a mammalian zygote using a method of the present disclosure can comprise a nucleotide sequence in some cases does not comprises a nucleotide sequence that encodes a polypeptide or a functional RNA. A polynucleotide to be delivered into a mammalian zygote using a method of the present disclosure can be an siRNA, a microRNA, an antisense RNA, an shRNA, a modified RNA, an antagomir RNA, or a DNA nucleic acid; an RNAi agent (e.g., an siRNA, an shRNA, etc.); an antisense RNA; an antisense oligonucleotide (ODN), e.g., a synthetic ODN having chemical modifications from native nucleic acids; a nucleic acid construct that express an antisense molecule as RNA.

A polypeptide to be delivered into a mammalian zygote using a method of the present disclosure can be any of a variety of polypeptides, including, but not limited to, a therapeutic polypeptide; a transcription activator; a transcription repressor; a polypeptide that modulates development; etc.

A polypeptide to be delivered into a mammalian zygote using a method of the present disclosure can have a length of from about 10 amino acids to about 10,000 amino acids; e.g., from about 10 amino acids to about 100 amino acids, from 100 amino acids to about 500 amino acids, from about 500 amino acids to about 1,000 amino acids, from about 1,000 amino acids to about 2000 amino acids, from about 2000 amino acids to about 3000 amino acids, from about 3000 amino acids to about 4000 amino acids, from about 4000 amino acids to about 5000 amino acids, from about 5000 amino acids to about 7500 amino acids, or from about 7500 amino acids to about 10,000 amino acids. A polypeptide to be delivered into a mammalian zygote using a method of the present disclosure can be from 0.1 kDa to 1000 kDa, e.g., from about 0.1 kDa to 0.5 kDa, from 0.5 kDa to 1 kDa, from 1 kDa to 10 kDa, from 10 kDa to 50 kDa, from 50 kDa to 100 kDa, from 100 kDa to 200 kDa, from 200 kDa to 300 kDa, from 300 kDa to 400 kDa, from 400 kDa to 500 kDa, from 500 kDa to 750 kDa, from 750 kDa to 1000 kDa, or more than 1000 kDa.

A method of the present disclosure involves electroporating a polypeptide or a polynucleotide into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a composition comprising a polynucleotide, forming a zygote/polynucleotide composition (an “electroporation mixture”); and b) electroporating the zygote/polynucleotide composition with 2 pulses at 30 V, where each pulse is a 3-millisecond (msec) pulse, with a 1 msec interval between the 2 pulses. In some cases, at least 50%, at least 60%, at least 70%, at least 80%, at least 85%, at least 90%, at least 95%, or at least 98%, of the zygotes are viable after electroporation.

A method of the present disclosure involves electroporating a polypeptide or a polynucleotide into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a composition comprising a polynucleotide, forming a zygote/polynucleotide composition (an “electroporation mixture”); and b) electroporating the zygote/polynucleotide composition with a single pulse at 30 V, where the single pulse is a 3-millisecond (msec) pulse. In some cases, at least 50%, at least 60%, at least 70%, at least 80%, at least 85%, at least 90%, at least 95%, or at least 98%, of the zygotes are viable after electroporation.

In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a composition comprising a polypeptide, forming a zygote/polypeptide composition (an “electroporation mixture”); and b) electroporating the zygote/polypeptide composition with 2 pulses at 30 V, where each pulse is a 3-millisecond (msec) pulse, with a 1 msec interval between the 2 pulses. In some cases, at least 50%, at least 60%, at least 70%, at least 80%, at least 85%, at least 90%, at least 95%, or at least 98%, of the zygotes are viable after electroporation.

In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a composition comprising a polypeptide, forming a zygote/polypeptide composition (an “electroporation mixture”); and b) electroporating the zygote/polypeptide composition with a single pulse at 30 V, where the single pulse is a 3-millisecond (msec) pulse. In some cases, at least 50%, at least 60%, at least 70%, at least 80%, at least 85%, at least 90%, at least 95%, or at least 98%, of the zygotes are viable after electroporation.

In some cases, electroporation of the zygote/polypeptide composition includes electroporating with one or more pulses. In some cases, electroporation of the zygote/polypeptide composition includes electroporating with multiple pulses. In some cases, electroporation of the zygote/polypeptide composition includes electroporating with 3 pulses. In some cases, electroporation of the zygote/polypeptide composition includes electroporating with 4 pulses. In some cases, electroporation of the zygote/polypeptide composition includes electroporating with 5 pulses. In some cases, electroporation of the zygote/polypeptide composition includes electroporating with 6 pulses. In some cases, electroporation of the zygote/polypeptide composition includes electroporating with 7 pulses. In some cases, electroporation of the zygote/polypeptide composition includes electroporating with 8 pulses. In some cases, electroporation of the zygote/polypeptide composition includes electroporating with 9 pulses. In some cases, electroporation of the zygote/polypeptide composition includes electroporating with 10 pulses. In some cases, at least 20%, at least 30%, at least 40%, at least 50%, at least 60%, at least 70%, at least 80%, at least 85%, at least 90%, at least 95%, or at least 98%, of the zygotes are viable after electroporation. In some cases, from 20% to 50% of the zygotes are viable after electroporation. In some cases, from 50% to 95% of the zygotes are viable after electroporation. In some cases, from 60% to 95% of the zygotes are viable after electroporation. In some cases, from 70% to 95% of the zygotes are viable after electroporation. In some cases, from 80% to 95% of the zygotes are viable after electroporation. In some cases, 100% of the zygotes are viable after electroporation.

In some cases, electroporation of the zygote/polypeptide composition includes electroporating with one or more pulses, where each pulse is a 1-millisecond (msec) pulse. In some cases, electroporation of the zygote/polypeptide composition includes electroporating with one or more pulses, where each pulse is a 2-millisecond (msec) pulse. In some cases, electroporation of the zygote/polypeptide composition includes electroporating with one or more pulses, where each pulse is a 3-millisecond (msec) pulse. In some cases, electroporation of the zygote/polypeptide composition includes electroporating with one or more pulses, where each pulse is a 4-millisecond (msec) pulse. In some cases, electroporation of the zygote/polypeptide composition includes electroporating with one or more pulses, where each pulse is a 3-millisecond (msec) pulse. In some cases, electroporation of the zygote/polypeptide composition includes electroporating with one or more pulses, where each pulse is a 5-millisecond (msec) pulse. In some cases, electroporation of the zygote/polypeptide composition includes electroporating with one or more pulses, where each pulse is a 6-millisecond (msec) pulse. In some cases, electroporation of the zygote/polypeptide composition includes electroporating with one or more pulses, where each pulse is a 7-millisecond pulse. In some cases, electroporation of the zygote/polypeptide composition includes electroporating with one or more pulses, where each pulse is an 8-millisecond pulse. In some cases, electroporation of the zygote/polypeptide composition includes electroporating with one or more pulses, where each pulse is a 9-millisecond pulse. In some cases, electroporation of the zygote/polypeptide composition includes electroporating with one or more pulses, where each pulse is a 10-millisecond pulse.

In some cases, electroporation of the zygote/polypeptide composition includes electroporating with multiple pulses at 10 V. In some cases, electroporation of the zygote/polypeptide composition includes electroporating with multiple pulses at 15 V. In some cases, electroporation of the zygote/polypeptide composition includes electroporating with multiple pulses at 20 V. In some cases, electroporation of the zygote/polypeptide composition includes electroporating with multiple pulses at 25 V. In some cases, electroporation of the zygote/polypeptide composition includes electroporating with multiple pulses at 30 V. In some cases, electroporation of the zygote/polypeptide composition includes electroporating with multiple pulses at 35 V. In some cases, electroporation of the zygote/polypeptide composition includes electroporating with multiple pulses at 40 V. In some cases, electroporation of the zygote/polypeptide composition includes electroporating with multiple pulses at 45 V. In some cases, electroporation of the zygote/polypeptide composition includes electroporating with multiple pulses at 50 V. In some cases, electroporation of the zygote/polypeptide composition includes electroporating with multiple pulses at 55 V. In some cases, electroporation of the zygote/polypeptide composition includes electroporating with multiple pulses at 60 V.

In some cases, electroporation of the zygote/polypeptide composition includes electroporating with 2 pulses at 30 volts, where each pulse is a 3-millisecond (msec) pulse. In some cases, electroporation of the zygote/polypeptide composition includes electroporating with 4 pulses at 30 volts, where each pulse is a 3-millisecond (msec) pulse. In some cases, electroporation of the zygote/polypeptide composition includes electroporating with 6 pulses at 30 volts, where each pulse is a 3-millisecond (msec) pulse. In some cases, electroporation of the zygote/polypeptide composition includes electroporating with 8 pulses at 30 volts, where each pulse is a 3-millisecond (msec) pulse. In some cases, electroporation of the zygote/polypeptide composition includes electroporating with 10 pulses at 30 volts, where each pulse is a 3-millisecond (msec) pulse. In some cases, electroporation of the zygote/polypeptide composition includes electroporating with 12 pulses at 30 volts, where each pulse is a 3-millisecond (msec) pulse.

A method of the present disclosure for delivering a polypeptide or a polynucleotide into a mammalian zygote can be used to deliver a polypeptide or a polynucleotide into any of a variety of mammalian zygotes, including, e.g., a human zygote or a non-human mammalian zygote. Non-human mammalian zygotes include, but are not limited to, a rodent zygote (e.g., a rat zygote; a mouse zygote); a lagomorph zygote (e.g., a rabbit zygote); a feline zygote, e.g., a cat zygote; a canine zygote, e.g., a dog zygote; an ovine (e.g., sheep) zygote; a caprine (e.g., goat) zygote; an equine (e.g., horse) zygote; an ungulate zygote; a non-human primate zygote; etc.

### Modifying Genomic DNA, Labeling Genomic DNA, Regulating Transcription in a Mammalian Zygote

The present disclosure provides methods of modifying the genome of a mammalian zygote. Methods of the present disclosure generally involve introducing a genome editing composition into a zygote via electroporation, where the genome editing composition comprises: i) a CRISPR/Cas endonuclease (or a nucleic acid comprising a nucleotide sequence encoding the CRISPR/Cas endonuclease); and ii) a corresponding guide RNA (or a nucleic acid comprising a nucleotide sequence encoding the guide RNA). In some cases, the genome editing composition comprises: i) a CRISPR/Cas endonuclease (or a nucleic acid comprising a nucleotide sequence encoding the CRISPR/Cas endonuclease); ii) a corresponding guide RNA (or a nucleic acid comprising a nucleotide sequence encoding the guide RNA); and iii) a donor DNA template (or a nucleic acid comprising a nucleotide sequence encoding the donor DNA template). In some cases, a method of the present disclosure comprises introducing into a mammalian zygote via electroporation a ribonucleoprotein (RNP) comprising a CRISPR/Cas endonuclease and a corresponding guide RNA. In some cases, a method of the present disclosure comprises introducing a genome-editing composition into a zygote via electroporation, where the genome editing composition comprises: a) an RNP comprising a CRISPR/Cas endonuclease and a corresponding guide RNA; and b) a donor DNA template. “Modifying” the genome is used herein interchangeably with “editing” the genome.

A method of the present disclosure for modifying the genome of a mammalian zygote can be used to modify the genome of any of a variety of mammalian zygotes, including, e.g., a human zygote or a non-human mammalian zygote. Non-human mammalian zygotes include, but are not limited to, a rodent zygote (e.g., a rat zygote; a mouse zygote); a lagomorph zygote (e.g., a rabbit zygote); a feline zygote, e.g., a cat zygote; a canine zygote, e.g., a dog zygote; an ovine (e.g., sheep) zygote; a caprine (e.g., goat) zygote; an equine (e.g., horse) zygote; an ungulate zygote; a non-human primate zygote; etc.

Genome editing includes non-homologous end joining (NHEJ) and homology-directed repair (HDR). A genome-editing endonuclease generates a single- or double-strand break in a target genomic DNA, and the single- or double-strand break is repaired. Repair that occurs via NHEJ is sometimes referred to an “indel” (insertion or deletion); DNA repair via HDR is sometimes referred to as “gene correction” or “gene modification.” In some cases, editing a target genomic DNA involves generating a substitution of one or more nucleotides in the target genomic DNA, generating an edited target genomic DNA. In some cases, editing a target genomic DNA involves deletion of one or more nucleotides from the target genomic DNA, generating an edited target genomic DNA. In some cases, editing a target genomic DNA involves insertion of one or more nucleotides from the target genomic DNA, generating an edited target genomic DNA.

A method of the present disclosure for modifying the genome of a zygote will in some cases result in NHEJ. Where a method of the present disclosure results in NHEJ, in some cases, a method of the present disclosure provides for an efficiency of NHEJ of at least 50%, at least 60%, at least 70%, at least 75%, at least 80%, at least 85%, at least 90%, at least 95%, or 100%. For example, where a plurality of zygotes are electroporated together with an RNP complex in an electroporation mixture, at least 50%, at least 60%, at least 70%, at least 75%, at least 80%, at least 85%, at least 90%, at least 95%, or 100%, of the zygotes will undergo NHEJ.

A method of the present disclosure for modifying the genome of a zygote will in some cases result in HDR. Where a method of the present disclosure results in HDR, in some cases, a method of the present disclosure provides for an efficiency of HDR of at least 15%, at least 20%, at least 25%, at least 30%, at least 35%, at least 40%, at least 45%, at least 50%, or more than 50%. For example, where a plurality of zygotes are electroporated together with an RNP complex and a donor DNA template in an electroporation mixture, at least 15%, at least 20%, at least 25%, at least 30%, at least 35%, at least 40%, at least 45%, at least 50%, or more than 50%, of the zygotes will undergo HDR.

The present disclosure provides methods of modulating transcription in a mammalian zygote. The methods generally involve introducing into the mammalian zygote an RNP complex comprising an enzymatically inactive CRISPR/Cas endonuclease (also referred to as a “dead Cas9” or “dCas9”) and a corresponding guide RNA. The enzymatically inactive CRISPR/Cas endonuclease retains the ability to bind to a target DNA when complexed with a guide RNA comprising a nucleotide sequence that is complementary to a nucleotide sequence in the target DNA; however, the enzymatically inactive CRISPR/Cas endonuclease does not cleave the target DNA.

The present disclosure provides methods of labeling a target nucleic acid in the genome of a mammalian zygote. The methods generally involve introducing into the mammalian zygote an RNP complex comprising: a) an enzymatically inactive CRISPR/Cas endonuclease (also referred to as a “dead Cas9” or “dCas9”); or a “nickase” CRISPR/Cas endonuclease (e.g., Cas9 D10A); and b) a corresponding guide RNA. In some cases, the CRISPR/Cas endonuclease comprises a detectable label, e.g., a fluorescent label. See, e.g., Deng et al. (2015) Proc. Natl. Acad. Sci. USA 112:11870. In some cases, the CRISPR/Cas endonuclease is a nickase, and the method is carried out in the presence of fluorescently labeled nucleotides. See, e.g., McCaffrey et al. (2016) Nucl. Acids Res. 44:e11.

A method of the present disclosure involves electroporating a ribonucleoprotein (RNP) complex into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a genome targeting composition, forming a zygote/genome targeting composition; and b) electroporating the zygote/genome targeting composition with 2 pulses at 30 V, where each pulse is a 3-millisecond (msec) pulse, with a 1 msec interval between the 2 pulses. In some cases, at least 50%, at least 60%, at least 70%, at least 80%, at least 85%, at least 90%, at least 95%, or at least 98%, of the zygotes are viable after electroporation. In some cases, from 50% to 95% of the zygotes are viable after electroporation. In some cases, from 60% to 95% of the zygotes are viable after electroporation. In some cases, from 70% to 95% of the zygotes are viable after electroporation. In some cases, from 80% to 95% of the zygotes are viable after electroporation. In some cases, 100% of the zygotes are viable after electroporation. In some cases, the genomic modification occurs via HDR or NHEJ. In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

A method of the present disclosure involves electroporating a ribonucleoprotein (RNP) complex into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a genome targeting composition, forming a zygote/genome targeting composition; and b) electroporating the zygote/genome targeting composition with a single pulse at 30 V, where the single pulse is a 3-millisecond (msec) pulse. In some cases, at least 50%, at least 60%, at least 70%, at least 80%, at least 85%, at least 90%, at least 95%, or at least 98%, of the zygotes are viable after electroporation. In some cases, from 50% to 95% of the zygotes are viable after electroporation. In some cases, from 60% to 95% of the zygotes are viable after electroporation. In some cases, from 70% to 95% of the zygotes are viable after electroporation. In some cases, from 80% to 95% of the zygotes are viable after electroporation. In some cases, 100% of the zygotes are viable after electroporation. In some cases, the genomic modification occurs via HDR or NHEJ. In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of an RNP complex, forming a zygote/RNP complex composition (an “electroporation mixture” or an “electroporation composition”); and b) electroporating the zygote/RNP complex composition with a single pulse at 30 V, where the single pulse is a 3-msec pulse. In some cases, the RNP is present in the electroporation composition at a concentration of from 5 μM to 16 μM. In some cases, the RNP is present in the electroporation composition at a concentration of 8 μM. In some cases, at least 50%, at least 60%, at least 70%, at least 80%, at least 85%, at least 90%, at least 95%, or at least 98%, of the zygotes are viable after electroporation with the RNP. In some cases, from 50% to 95% of the zygotes are viable after electroporation with the RNP. In some cases, from 60% to 95% of the zygotes are viable after electroporation with the RNP. In some cases, from 70% to 95% of the zygotes are viable after electroporation with the RNP. In some cases, from 80% to 95% of the zygotes are viable after electroporation with the RNP. In some cases, 100% of the zygotes are viable after electroporation with the RNP. In some cases, the genomic modification occurs via homology-directed repair (HDR) or non-homologous end joining (NHEJ). In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

In some cases, the RNP complex comprises an RNA and a DNA-binding polypeptide, where the RNA and the DNA-binding polypeptide are present in a ratio of from 0.5:1 to 1:1, from 1:1 to 1:1.5, or from 1:1.5 to 1:2 RNA:DNA-binding polypeptide. In some cases, the RNP complex is present in the electroporation mixture at a concentration of from 5 μM to 15 μM, e.g., from 5 μM to 10 μM, or from 10 μM to 15 μM. In some cases, the RNP complex is present in the electroporation mixture at a concentration of 8 μM. In some cases, the electroporation mixture includes a donor DNA template. The donor DNA template can be part of the RNP, or can be separate from the RNP.

In some cases, from 1 to 10 pulses of 30 V each are applied. In some cases, a single pulse of 30 V is applied. In some cases, 2 pulses of 30 V each are applied. In some cases, 3 pulses of 30 V each are applied. In some cases, 4 pulses of 30 V each are applied. In some cases, 5 pulses of 30 V each are applied. In some cases, 6 pulses of 30 V each are applied. In some cases, 7 pulses of 30 V each are applied. In some cases, 8 pulses of 30 V each are applied. In some cases, 9 pulses of 30 V each are applied. In some cases, 10 pulses of 30 V each are applied. Each pulse can be from 1 millisecond to 10 milliseconds in duration. In some cases, each pulse is a 1-millisecond pulse. In some cases, each pulse is a 2-millisecond pulse. In some cases, each pulse is a 3-millisecond pulse. In some cases, each pulse is a 4-millisecond pulse. In some cases, each pulse is a 5-millisecond pulse. In some cases, each pulse is a 6-millisecond pulse. In some cases, each pulse is a 7-millisecond pulse. In some cases, each pulse is an 8-millisecond pulse. In some cases, each pulse is a 9-millisecond pulse. In some cases, each pulse is a 10-millisecond pulse. In some case, 6 pulses of 30 V per pulse are applied, where each pulse is a 3-millisecond pulse.

### Genome Targeting Compositions

A genome targeting composition is a composition that includes a genome editing nuclease that is (or can be) targeted to a desired sequence within a target genome.

Examples of suitable genome editing nucleases are CRISPR/Cas endonucleases (e.g., class 2 CRISPR/Cas endonucleases such as a type II, type V, or type VI CRISPR/Cas endonucleases). Thus, a genome targeting composition can include a CRISPR/Cas endonuclease (e.g., a class 2 CRISPR/Cas endonuclease such as a type II, type V, or type VI CRISPR/Cas endonuclease). In some cases, a genome targeting composition includes a class 2 CRISPR/Cas endonuclease. In some cases, a genome targeting composition includes a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a genome targeting composition includes a class 2 type VI CRISPR/Cas endonuclease (e.g., a C2c2 protein).

As described in more detail below, a CRISPR/Cas endonuclease interacts with (binds to) a corresponding guide RNA to form a ribonucleoprotein (RNP) complex that is targeted to a particular site in a target genome via base pairing between the guide RNA and a target sequence within the target genome. A guide RNA includes a nucleotide sequence (a guide sequence) that is complementary to a sequence (the target site) of a target nucleic acid. Thus, when a subject genome targeting composition includes a CRISPR/Cas endonuclease (e.g., a class 2 CRISPR/Cas endonuclease), it must also include a corresponding guide RNA when being used in a method to cleave a target DNA. However, because the guide RNA can be readily modified in order to target any desired sequence within a target genome, in some cases, a composition includes only the CRISPR/Cas endonuclease (or a nucleic acid encoding the CRISPR/Cas endonuclease) until a user adds the desired corresponding guide RNA (or a nucleic acid encoding the corresponding guide RNA).

The components of a genome targeting composition can be delivered (introduced into a zygote) as DNA, RNA, or protein. For example, when the composition includes a class 2 CRISPR/Cas endonuclease (e.g., Cas9, Cpf1, etc.) and a corresponding guide RNA (e.g., a Cas9 guide RNA, a Cpf1 guide RNA, etc.), the endonuclease and guide RNA can be delivered (introduced into the zygote) as an RNP complex (i.e., a pre-assembled complex of the CRISPR/Cas endonuclease and the corresponding CRISPR/Cas guide RNA). Thus, a class 2 CRISPR/Cas endonuclease can be introduced into a zygote as a protein. Alternatively, a class 2 CRISPR/Cas endonuclease can be introduced into a zygote as a nucleic acid (DNA and/or RNA) encoding the endonuclease. A CRISPR/Cas guide RNA can be introduced into a zygote as RNA, or as DNA encoding the guide RNA.

In some cases, a genome editing nuclease is a fusion protein that is fused to a heterologous polypeptide (also referred to as a “fusion partner”). In some cases, a genome editing nuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.). In some embodiments, a genome editing nuclease is fused to an amino acid sequence (a fusion partner) that provides a tag (i.e., the fusion partner is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some embodiments, the fusion partner can provide for increased or decreased stability (i.e., the fusion partner can be a stability control peptide, e.g., a degron, which in some cases is controllable (e.g., a temperature sensitive or drug controllable degron sequence).

In some cases, a genome editing nuclease is conjugated (e.g., fused) to a polypeptide permeant domain to promote uptake by the zygote (i.e., the fusion partner promotes uptake by a cell). A number of permeant domains are known in the art and may be used, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 1080). As another example, the permeant peptide can comprise the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site can be determined by routine experimentation.

In some cases, a genome editing nuclease includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus a polypeptide (e.g., a genome editing nuclease, e.g., a Cas9 protein). In some embodiments, a PTD is covalently linked to the carboxyl terminus of a polypeptide (e.g., a genome editing nuclease, e.g., a Cas9 protein). In some cases, the PTD is inserted internally in the genome editing nuclease (e.g., Cas9 protein) (i.e., is not at the N- or C-terminus of the genome editing nuclease). In some cases, a subject genome editing nuclease (e.g., Cas9 protein) includes (is conjugated to, is fused to) one or more PTDs (e.g., two or more, three or more, four or more PTDs). In some cases a PTD includes a nuclear localization signal (NLS) (e.g., in some cases 2 or more, 3 or more, 4 or more, or 5 or more NLSs).

In some cases, a genome editing nuclease (e.g., Cas9 protein) includes one or more NLSs (e.g., 2 or more, 3 or more, 4 or more, or 5 or more NLSs). In some embodiments, a PTD is covalently linked to a nucleic acid (e.g., a CRISPR/Cas guide RNA, a polynucleotide encoding a CRISPR/Cas guide RNA, a polynucleotide encoding a class 2 CRISPR/Cas endonuclease such as a Cas9 protein or a type V or type VI CRISPR/Cas protein, etc.). Examples of PTDs include but are not limited to a minimal undecapeptide protein transduction domain (corresponding to residues 47-57 of HIV-1 TAT comprising YGRKKRRQRRR; SEQ ID NO: 1076); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:1077); Transportan GWTLNSAGYLLGKINLKALAALAKKIL (SEQ ID NO:1078); KALAWEAKLAKALAKALAKHLAKALAKALKCEA (SEQ ID NO:1079); and RQIKIWFQNRRMKWKK (SEQ ID NO: 1080). Exemplary PTDs include but are not limited to, YGRKKRRQRRR (SEQ ID NO:1081), RKKRRQRRR (SEQ ID NO:1082); an arginine homopolymer of from 3 arginine residues to 50 arginine residues; Exemplary PTD domain amino acid sequences include, but are not limited to, any of the following: YGRKKRRQRRR (SEQ ID NO:1083); RKKRRQRR (SEQ ID NO:1084); YARAAARQARA (SEQ ID NO:1085); THRLPRRRRRR (SEQ ID NO:1086); and GGRRARRRRRR (SEQ ID NO:1087). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

A genome editing nuclease (e.g., Cas9 protein) can have multiple (1 or more, 2 or more, 3 or more, etc.) fusion partners in any combination of the above. As an illustrative example, a genome editing nuclease (e.g., Cas9 protein) can have a fusion partner that provides for tagging (e.g., GFP), and can also have a subcellular localization sequence (e.g., one or more NLSs). In some cases, such a fusion protein might also have a tag for ease of tracking and/or purification (e.g., a histidine tag, e.g., a 6×His (His-His-His-His-His-His) tag; a hemagglutinin (HA) tag; a FLAG tag; a Myc tag; and the like). As another illustrative example, genome editing nuclease (e.g., Cas9 protein) can have one or more NLSs (e.g., two or more, three or more, four or more, five or more, 1, 2, 3, 4, or 5 NLSs). In some cases a fusion partner (or multiple fusion partners, e.g., 1, 2, 3, 4, or 5 NLSs) (e.g., an NLS, a tag, a fusion partner providing an activity, etc.) is located at or near the C-terminus of the genome editing nuclease (e.g., Cas9 protein). In some cases a fusion partner (or multiple fusion partners, e.g., 1, 2, 3, 4, or 5 NLSs) (e.g., an NLS, a tag, a fusion partner providing an activity, etc.) is located at the N-terminus of the genome editing nuclease (e.g., Cas9 protein). In some cases the genome editing nuclease (e.g., Cas9 protein) has a fusion partner (or multiple fusion partners, e.g., 1, 2, 3, 4, or 5 NLSs)(e.g., an NLS, a tag, a fusion partner providing an activity, etc.) at both the N-terminus and C-terminus.

### Class 2 CRISPR/Cas Endonucleases

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In some embodiments, a genome editing nuclease of a genome targeting composition of the present disclosure is a class 2 CRISPR/Cas endonuclease. Thus in some cases, a subject genome targeting composition includes a class 2 CRISPR/Cas endonuclease (or a nucleic encoding the endonuclease). In class 2 CRISPR systems, the functions of the effector complex (e.g., the cleavage of target DNA) are carried out by a single endonuclease (e.g., see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97). As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9), type V CRISPR/Cas proteins (e.g., Cpf1, C2c1, C2C3), and type VI CRISPR/Cas proteins (e.g., C2c2). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

### Type II CRISPR/Cas Endonucleases (e.g., Cas 9)

In natural Type II CRISPR/Cas systems, Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual- (dgRNA) or single guide RNA (sgRNA) form a ribonucleoprotein (RNP) complex that can be targeted to a desired DNA sequence. Guided by a dual-RNA complex or a chimeric single-guide RNA, Cas9 generates site-specific DSBs or SSBs within double-stranded DNA (dsDNA) target nucleic acids, which are repaired either by non-homologous end joining (NHEJ) or homology-directed recombination (HDR).

As noted above, in some cases, a genome targeting composition of the present disclosure includes a type II CRISPR/Cas endonuclease. A type II CRISPR/Cas endonuclease is a type of class 2 CRISPR/Cas endonuclease. In some cases, the type II CRISPR/Cas endonuclease is a Cas9 protein. A Cas9 protein forms a complex with a Cas9 guide RNA. The guide RNA provides target specificity to a Cas9-guide RNA complex by having a nucleotide sequence (a guide sequence) that is complementary to a sequence (the target site) of a target nucleic acid (as described elsewhere herein). The Cas9 protein of the complex provides the site-specific activity. In other words, the Cas9 protein is guided to a target site (e.g., stabilized at a target site) within a target nucleic acid sequence (e.g. a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA.

A Cas9 protein can bind and/or modify (e.g., cleave, nick, methylate, demethylate, etc.) a target nucleic acid and/or a polypeptide associated with target nucleic acid (e.g., methylation or acetylation of a histone tail)(e.g., when the Cas9 protein includes a fusion partner with an activity). In some cases, the Cas9 protein is a naturally-occurring protein (e.g., naturally occurs in bacterial and/or archaeal cells). In other cases, the Cas9 protein is not a naturally-occurring polypeptide (e.g., the Cas9 protein is a variant Cas9 protein, a chimeric protein, and the like).

Examples of suitable Cas9 proteins include, but are not limited to, those set forth in SEQ ID NOs: 5-816. Naturally occurring Cas9 proteins bind a Cas9 guide RNA, are thereby directed to a specific sequence within a target nucleic acid (a target site), and cleave the target nucleic acid (e.g., cleave dsDNA to generate a double strand break, cleave ssDNA, cleave ssRNA, etc.). A chimeric Cas9 protein is a fusion protein comprising a Cas9 polypeptide that is fused to a heterologous protein (referred to as a fusion partner), where the heterologous protein provides an activity (e.g., one that is not provided by the Cas9 protein). The fusion partner can provide an activity, e.g., enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc.). In some cases a portion of the Cas9 protein (e.g., the RuvC domain and/or the HNH domain) exhibits reduced nuclease activity relative to the corresponding portion of a wild type Cas9 protein (e.g., in some cases the Cas9 protein is a nickase). In some cases, the Cas9 protein is enzymatically inactive, or has reduced enzymatic activity relative to a wild-type Cas9 protein (e.g., relative to Streptococcus pyogenes Cas9).

Assays to determine whether given protein interacts with a Cas9 guide RNA can be any convenient binding assay that tests for binding between a protein and a nucleic acid. Suitable binding assays (e.g., gel shift assays) will be known to one of ordinary skill in the art (e.g., assays that include adding a Cas9 guide RNA and a protein to a target nucleic acid).

Assays to determine whether a protein has an activity (e.g., to determine if the protein has nuclease activity that cleaves a target nucleic acid and/or some heterologous activity) can be any convenient assay (e.g., any convenient nucleic acid cleavage assay that tests for nucleic acid cleavage). Suitable assays (e.g., cleavage assays) will be known to one of ordinary skill in the art and can include adding a Cas9 guide RNA and a protein to a target nucleic acid.

In some cases, a chimeric Cas9 protein includes a heterologous polypeptide that has enzymatic activity that modifies target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other cases, a chimeric Cas9 protein includes a heterologous polypeptide that has enzymatic activity that modifies a polypeptide (e.g., a histone) associated with target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Many Cas9 orthologs from a wide variety of species have been identified and in some cases the proteins share only a few identical amino acids. Identified Cas9 orthologs have similar domain architecture with a central HNH endonuclease domain and a split RuvC/RNaseH domain (e.g., RuvCI, RuvCII, and RuvCIII) (e.g., see Table 1). For example, a Cas9 protein can have 3 different regions (sometimes referred to as RuvC-I, RuvC-II, and RucC-III), that are not contiguous with respect to the primary amino acid sequence of the Cas9 protein, but fold together to form a RuvC domain once the protein is produced and folds. Thus, Cas9 proteins can be said to share at least 4 key motifs with a conserved architecture. Motifs 1, 2, and 4 are RuvC like motifs while motif 3 is an HNH-motif. The motifs set forth in Table 1 may not represent the entire RuvC-like and/or HNH domains as accepted in the art, but Table 1 does present motifs that can be used to help determine whether a given protein is a Cas9 protein.

TABLE 1
Table 1 lists 4 motifs that are present in Cas9 sequences from various
species. The amino acids listed in Table 1 are from the Cas9 from S.
pyogenes (SEQ ID NO: 5).

| Motif # | Motif | Amino acids (residue #s) | Highly conserved |
|---|---|---|---|
| 1 | RuvC-like I | IGLDIGTNSVGWAVI (7-21) | D10, G12, G17 |
| | | (SEQ ID NO: 1) | |
| 2 | RuvC-like II | IVIEMARE (759-766) | E762 |
| | | (SEQ ID NO: 2) | |
| 3 | HNH-motif | DVDHIVPQSFLKDDSIDNKVLTRSDK | H840, N854, N863 |
| | | N (837-863) (SEQ ID NO: 3) | |
| 4 | RuvC-like | HHAHDAYL (982-989) | H982, H983, A984, |
| | III | (SEQ ID NO: 4) | D986, A987 |

In some cases, a suitable Cas9 protein comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 as set forth in SEQ ID NOs: 1-4, respectively (e.g., see Table 1), or to the corresponding portions in any of the amino acid sequences set forth in SEQ ID NOs: 5-816.

In other words, in some cases, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 of the Cas9 amino acid sequence set forth in SEQ ID NO: 5 (e.g., the sequences set forth in SEQ ID NOs: 1-4, e.g., see Table 1), or to the corresponding portions in any of the amino acid sequences set forth in SEQ ID NOs: 6-816.

In some cases, a suitable Cas9 protein comprises an amino acid sequence having 4 motifs, each of motifs 1-4 having 60% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 70% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 75% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 80% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 85% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 90% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 95% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 99% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 100% amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816.

In some cases, a suitable Cas9 protein comprises an amino acid sequence having 60% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 70% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 75% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 80% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 85% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 90% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 95% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 99% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 100% amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to the Cas9 amino acid sequence set forth in SEQ ID NO: 5, or to any of the amino acid sequences set forth as SEQ ID NOs: 6-816.

In some cases, a suitable Cas9 protein comprises an amino acid sequence having 60% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 70% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 75% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 80% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 85% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 90% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 95% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 99% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 100% amino acid sequence identity to the Cas9 amino acid sequence set forth in SEQ ID NO: 5, or to any of the amino acid sequences set forth as SEQ ID NOs: 6-816. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a Cas9 protein comprises 4 motifs (as listed in Table 1), at least one with (or each with) amino acid sequences having 75% or more, 80% or more, 85% or more, 90% or more, 95% or more, 99% or more or 100% amino acid sequence identity to each of the 4 motifs listed in Table 1 (SEQ ID NOs: 1-4), or to the corresponding portions in any of the amino acid sequences set forth as SEQ ID NOs: 6-816.

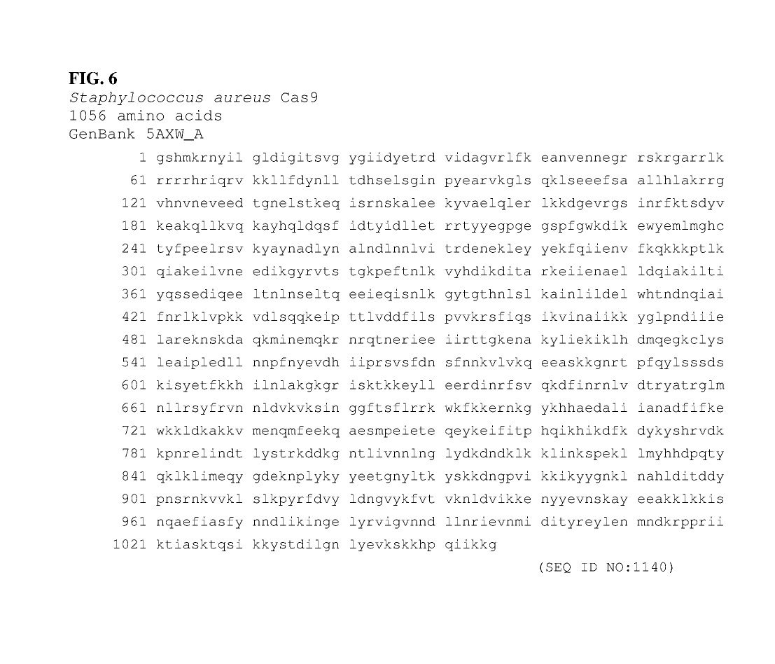

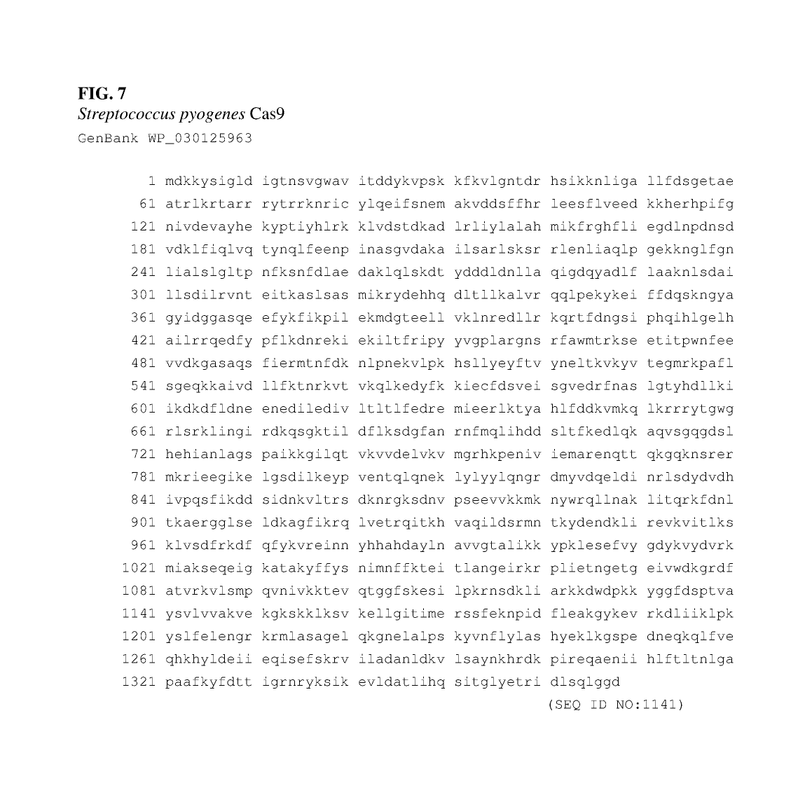

In some cases, the Cas9 polypeptide used in a composition or method of the present disclosure is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 6 (SEQ ID NO: 1140).

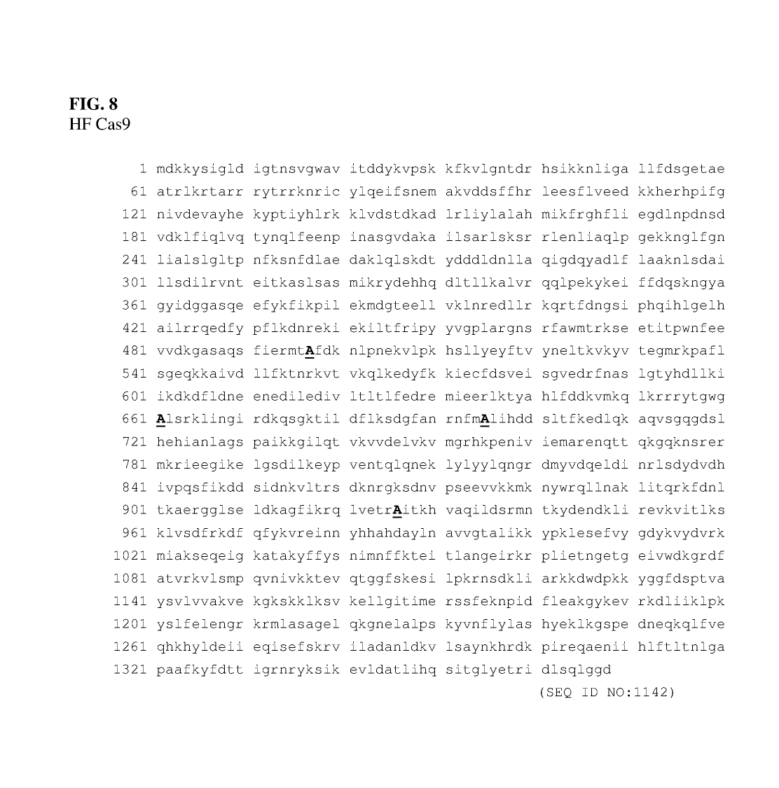

In some cases, the Cas9 polypeptide used in a composition or method of the present disclosure is comprises an amino acid sequence having at least 50%, at least 55%, at least 60%, at least 65%, at least 70%, at least 75%, or at least 80%, amino acid sequence identity to the Streptococcus pyogenes Cas9 amino acid sequence depicted in FIG. 7 (SEQ ID NO:1141). In some cases, the Cas9 polypeptide used in a composition or method of the present disclosure is comprises an amino acid sequence having at least 85%, at least 90%, at least 95%, at least 98%, at least 99%, or 100%, amino acid sequence identity to the Streptococcus pyogenes Cas9 amino acid sequence depicted in FIG. 7 (SEQ ID NO:1141).

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 7 (SEQ ID NO:1141) are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 7 (SEQ ID NO:1141), where amino acids N497, R661, Q695, and Q926 are substituted, e.g., with alanine. For example, in some cases, an HF Cas9 polypeptide comprised the amino acid sequence depicted in FIG. 8 (SEQ ID NO: 1142).

In some cases, a suitable Cas9 polypeptide exhibits altered PAM specificity. See, e.g., Kleinstiver et al. (2015) Nature 523:481.

### Type V and Type VI CRISPR/Cas Endonucleases

In some cases, a genome targeting composition of the present disclosure includes a type V or type VI CRISPR/Cas endonuclease (i.e., the genome editing endonuclease is a type V or type VI CRISPR/Cas endonuclease) (e.g., Cpf1, C2c1, C2c2, C2c3). Type V and type VI CRISPR/Cas endonucleases are a type of class 2 CRISPR/Cas endonuclease. Examples of type V CRISPR/Cas endonucleases include but are not limited to: Cpf1, C2c1, and C2c3. An example of a type VI CRISPR/Cas endonuclease is C2c2. In some cases, a subject genome targeting composition includes a type V CRISPR/Cas endonuclease (e.g., Cpf1, C2c1, C2c3). In some cases, a Type V CRISPR/Cas endonuclease is a Cpf1 protein. In some cases, a subject genome targeting composition includes a type VI CRISPR/Cas endonuclease (e.g., C2c2).

Like type II CRISPR/Cas endonucleases, type V and VI CRISPR/Cas endonucleases form a complex with a corresponding guide RNA. The guide RNA provides target specificity to an endonuclease-guide RNA RNP complex by having a nucleotide sequence (a guide sequence) that is complementary to a sequence (the target site) of a target nucleic acid (as described elsewhere herein). The endonuclease of the complex provides the site-specific activity. In other words, the endonuclease is guided to a target site (e.g., stabilized at a target site) within a target nucleic acid sequence (e.g. a chromosomal sequence or an extrachromosomal sequence, e.g., an episomal sequence, a minicircle sequence, a mitochondrial sequence, a chloroplast sequence, etc.) by virtue of its association with the protein-binding segment of the guide RNA.

Examples and guidance related to type V and type VI CRISPR/Cas proteins (e.g., cpf1, C2c1, C2c2, and C2c3 guide RNAs) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97.

In some cases, the Type V or type VI CRISPR/Cas endonuclease (e.g., Cpf1, C2c1, C2c2, C2c3) is enzymatically active, e.g., the Type V or type VI CRISPR/Cas polypeptide, when bound to a guide RNA, cleaves a target nucleic acid. In some cases, the Type V or type VI CRISPR/Cas endonuclease (e.g., Cpf1, C2c1, C2c2, C2c3) exhibits reduced enzymatic activity relative to a corresponding wild-type a Type V or type VI CRISPR/Cas endonuclease (e.g., Cpf1, C2c1, C2c2, C2c3), and retains DNA binding activity.

In some cases a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs: 1088-1092. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the Cpf1 amino acid sequence set forth in any of SEQ ID NOs: 1088-1092.

In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the Cpf1 amino acid sequence set forth in any of SEQ ID NOs: 1088-1092. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the Cpf1 amino acid sequence set forth in any of SEQ ID NOs: 1088-1092. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the Cpf1 amino acid sequence set forth in any of SEQ ID NOs: 1088-1092. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the Cpf1 amino acid sequence set forth in any of SEQ ID NOs: 1088-1092.

In some cases, the Cpf1 protein exhibits reduced enzymatic activity relative to a wild-type Cpf1 protein (e.g., relative to a Cpf1 protein comprising the amino acid sequence set forth in any of SEQ ID NOs: 1088-1092), and retains DNA binding activity. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs: 1088-1092; and comprises an amino acid substitution (e.g., a D-A substitution) at an amino acid residue corresponding to amino acid 917 of the Cpf1 amino acid sequence set forth in SEQ ID NO: 1088. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs: 1088-1092; and comprises an amino acid substitution (e.g., an E→A substitution) at an amino acid residue corresponding to amino acid 1006 of the Cpf1 amino acid sequence set forth in SEQ ID NO: 1088. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs: 1088-1092; and comprises an amino acid substitution (e.g., a D→A substitution) at an amino acid residue corresponding to amino acid 1255 of the Cpf1 amino acid sequence set forth in SEQ ID NO: 1088.

In some cases, a suitable Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs: 1088-1092.

In some cases a type V CRISPR/Cas endonuclease is a C2c1 protein (examples include those set forth as SEQ ID NOs: 1112-1119). In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c1 amino acid sequence set forth in any of SEQ ID NOs: 1112-1119. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c1 amino acid sequence set forth in any of SEQ ID NOs: 1112-1119.

In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the C2c1 amino acid sequences set forth in any of SEQ ID NOs: 1112-1119). In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the C2c1 amino acid sequence set forth in any of SEQ ID NOs: 1112-1119. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the C2c1 amino acid sequence set forth in any of SEQ ID NOs: 1112-1119. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the C2c1 amino acid sequence set forth in any of SEQ ID NOs: 1112-1119.

In some cases, the C2c1 protein exhibits reduced enzymatic activity relative to a wild-type C2c1 protein (e.g., relative to a C2c1 protein comprising the amino acid sequence set forth in any of SEQ ID NOs: 1112-1119), and retains DNA binding activity. In some cases, a suitable C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c1 amino acid sequence set forth in any of SEQ ID NOs: 1112-1119.

In some cases a type V CRISPR/Cas endonuclease is a C2c3 protein (examples include those set forth as SEQ ID NOs: 1120-1123). In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c3 amino acid sequence set forth in any of SEQ ID NOs: 1120-1123. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c3 amino acid sequence set forth in any of SEQ ID NOs: 1120-1123.

In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the C2c3 amino acid sequence set forth in any of SEQ ID NOs: 1120-1123. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the C2c3 amino acid sequence set forth in any of SEQ ID NOs: 1120-1123. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the C2c3 amino acid sequence set forth in any of SEQ ID NOs: 1120-1123. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the C2c3 amino acid sequence set forth in any of SEQ ID NOs: 1120-1123.

In some cases, the C2c3 protein exhibits reduced enzymatic activity relative to a wild-type C2c3 protein (e.g., relative to a C2c3 protein comprising the amino acid sequence set forth in any of SEQ ID NOs: 1120-1123), and retains DNA binding activity. In some cases, a suitable C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c3 amino acid sequence set forth in any of SEQ ID NOs: 1120-1123.

In some cases a type VI CRISPR/Cas endonuclease is a C2c2 protein (examples include those set forth as SEQ ID NOs: 1124-1135). In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c2 amino acid sequence set forth in any of SEQ ID NOs: 1124-1135. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c2 amino acid sequence set forth in any of SEQ ID NOs: 1124-1135.

In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the C2c2 amino acid sequence set forth in any of SEQ ID NOs: 1124-1135. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the C2c2 amino acid sequence set forth in any of SEQ ID NOs: 1124-1135. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the C2c2 amino acid sequence set forth in any of SEQ ID NOs: 1124-1135. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the C2c2 amino acid sequence set forth in any of SEQ ID NOs: 1124-1135.

In some cases, the C2c2 protein exhibits reduced enzymatic activity relative to a wild-type C2c2 protein (e.g., relative to a C2c2 protein comprising the amino acid sequence set forth in any of SEQ ID NOs: 1124-1135), and retains DNA binding activity. In some cases, a suitable C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c2 amino acid sequence set forth in any of SEQ ID NOs: 1124-1135.

### Guide RNA (for CRISPR/Cas Endonucleases)

A nucleic acid molecule that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.”

A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

A guide RNA can be referred to by the protein to which it corresponds. For example, when the class 2 CRISPR/Cas endonuclease is a Cas9 protein, the corresponding guide RNA can be referred to as a “Cas9 guide RNA.” Likewise, as another example, when the class 2 CRISPR/Cas endonuclease is a Cpf1 protein, the corresponding guide RNA can be referred to as a “Cpf1 guide RNA.”

In some embodiments, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

### Cas9 Guide RNA

A nucleic acid molecule that binds to a Cas9 protein and targets the complex to a specific location within a target nucleic acid is referred to herein as a “Cas9 guide RNA.”

A Cas9 guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule.

The first segment (targeting segment) of a Cas9 guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide. The protein-binding segment of a subject Cas9 guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid.

A Cas9 guide RNA and a Cas9 protein form a complex (e.g., bind via non-covalent interactions). The Cas9 guide RNA provides target specificity to the complex by including a targeting segment, which includes a guide sequence (a nucleotide sequence that is complementary to a sequence of a target nucleic acid). The Cas9 protein of the complex provides the site-specific activity (e.g., cleavage activity or an activity provided by the Cas9 protein when the Cas9 protein is a Cas9 fusion polypeptide, i.e., has a fusion partner). In other words, the Cas9 protein is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the Cas9 guide RNA.

The “guide sequence” also referred to as the “targeting sequence” of a Cas9 guide RNA can be modified so that the Cas9 guide RNA can target a Cas9 protein to any desired sequence of any desired target nucleic acid, with the exception that the protospacer adjacent motif (PAM) sequence can be taken into account. Thus, for example, a Cas9 guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some embodiments, a Cas9 guide RNA includes two separate nucleic acid molecules: an “activator” and a “targeter” and is referred to herein as a “dual Cas9 guide RNA”, a “double-molecule Cas9 guide RNA”, or a “two-molecule Cas9 guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

A Cas9 guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator/tracrRNA) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the Cas9 guide RNA. As such, each targeter molecule can be said to have a corresponding activator molecule (which has a region that hybridizes with the targeter). The targeter molecule additionally provides the targeting segment. Thus, a targeter and an activator molecule (as a corresponding pair) hybridize to form a Cas9 guide RNA. The exact sequence of a given crRNA or tracrRNA molecule is characteristic of the species in which the RNA molecules are found. A subject dual Cas9 guide RNA can include any corresponding activator and targeter pair.

The term “activator” or “activator RNA” is used herein to mean a tracrRNA-like molecule (tracrRNA: “trans-acting CRISPR RNA”) of a Cas9 dual guide RNA (and therefore of a Cas9 single guide RNA when the “activator” and the “targeter” are linked together by, e.g., intervening nucleotides). Thus, for example, a Cas9 guide RNA (dgRNA or sgRNA) comprises an activator sequence (e.g., a tracrRNA sequence). A tracr molecule (a tracrRNA) is a naturally existing molecule that hybridizes with a CRISPR RNA molecule (a crRNA) to form a Cas9 dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a Cas9 dual guide RNA (and therefore of a Cas9 single guide RNA when the “activator” and the “targeter” are linked together, e.g., by intervening nucleotides). Thus, for example, a Cas9 guide RNA (dgRNA or sgRNA) comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

A Cas9 guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A Cas9 guide RNA (e.g. a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator) comprises a stretch of nucleotides (a duplex-forming segment) that forms the other half of the dsRNA duplex of the protein-binding segment of the Cas9 guide RNA. In other words, a stretch of nucleotides of the targeter is complementary to and hybridizes with a stretch of nucleotides of the activator to form the dsRNA duplex of the protein-binding segment of a Cas9 guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a Cas9 guide RNA. The particular sequence of a given naturally existing crRNA or tracrRNA molecule is characteristic of the species in which the RNA molecules are found. Examples of suitable activator and targeter are well known in the art.

A Cas9 guide RNA (e.g. a dual guide RNA or a single guide RNA) can be comprised of any corresponding activator and targeter pair. Non-limiting examples of nucleotide sequences that can be included in a Cas9 guide RNA (dgRNA or sgRNA) include sequences set forth in SEQ ID NOs: 827-1075, or complements thereof. For example, in some cases, sequences from SEQ ID NOs: 827-957 (which are from tracrRNAs) or complements thereof, can pair with sequences from SEQ ID NOs: 964-1075 (which are from crRNAs), or complements thereof, to form a dsRNA duplex of a protein binding segment.

### Targeting Segment of a Cas9 Guide RNA

The first segment of a subject guide nucleic acid includes a guide sequence (i.e., a targeting sequence)(a nucleotide sequence that is complementary to a sequence (a target site) in a target nucleic acid). In other words, the targeting segment of a subject guide nucleic acid can interact with a target nucleic acid (e.g., double stranded DNA (dsDNA)) in a sequence-specific manner via hybridization (i.e., base pairing). As such, the nucleotide sequence of the targeting segment may vary (depending on the target) and can determine the location within the target nucleic acid that the Cas9 guide RNA and the target nucleic acid will interact. The targeting segment of a Cas9 guide RNA can be modified (e.g., by genetic engineering)/designed to hybridize to any desired sequence (target site) within a target nucleic acid (e.g., a eukaryotic target nucleic acid such as genomic DNA).

The targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

The nucleotide sequence (the targeting sequence) of the targeting segment that is complementary to a nucleotide sequence (target site) of the target nucleic acid can have a length of 10 nt or more. For example, the targeting sequence of the targeting segment that is complementary to a target site of the target nucleic acid can have a length of 12 nt or more, 15 nt or more, 18 nt or more, 19 nt or more, or 20 nt or more. In some cases, the nucleotide sequence (the targeting sequence) of the targeting segment that is complementary to a nucleotide sequence (target site) of the target nucleic acid has a length of 12 nt or more. In some cases, the nucleotide sequence (the targeting sequence) of the targeting segment that is complementary to a nucleotide sequence (target site) of the target nucleic acid has a length of 18 nt or more.

For example, the targeting sequence of the targeting segment that is complementary to a target sequence of the target nucleic acid can have a length of from 10 to 100 nucleotides (nt) (e.g., from 10 to 90 nt, from 10 to 75 nt, from 10 to 60 nt, from 10 to 50 nt, from 10 to 35 nt, from 10 to 30 nt, from 10 to 25 nt, from 10 to 22 nt, from 10 to 20 nt, from 12 to 100 nt, from 12 to 90 nt, from 12 to 75 nt, from 12 to 60 nt, from 12 to 50 nt, from 12 to 35 nt, from 12 to 30 nt, from 12 to 25 nt, from 12 to 22 nt, from 12 to 20 nt, from 15 to 100 nt, from 15 to 90 nt, from 15 to 75 nt, from 15 to 60 nt, from 15 to 50 nt, from 15 to 35 nt, from 15 to 30 nt, from 15 to 25 nt, from 15 to 22 nt, from 15 to 20 nt, from 17 to 100 nt, from 17 to 90 nt, from 17 to 75 nt, from 17 to 60 nt, from 17 to 50 nt, from 17 to 35 nt, from 17 to 30 nt, from 17 to 25 nt, from 17 to 22 nt, from 17 to 20 nt, from 18 to 100 nt, from 18 to 90 nt, from 18 to 75 nt, from 18 to 60 nt, from 18 to 50 nt, from 18 to 35 nt, from 18 to 30 nt, from 18 to 25 nt, from 18 to 22 nt, or from 18 to 20 nt). In some cases, the targeting sequence of the targeting segment that is complementary to a target sequence of the target nucleic acid has a length of from 15 nt to 30 nt. In some cases, the targeting sequence of the targeting segment that is complementary to a target sequence of the target nucleic acid has a length of from 15 nt to 25 nt. In some cases, the targeting sequence of the targeting segment that is complementary to a target sequence of the target nucleic acid has a length of from 18 nt to 30 nt. In some cases, the targeting sequence of the targeting segment that is complementary to a target sequence of the target nucleic acid has a length of from 18 nt to 25 nt. In some cases, the targeting sequence of the targeting segment that is complementary to a target sequence of the target nucleic acid has a length of from 18 nt to 22 nt. In some cases, the targeting sequence of the targeting segment that is complementary to a target site of the target nucleic acid is 20 nucleotides in length. In some cases, the targeting sequence of the targeting segment that is complementary to a target site of the target nucleic acid is 19 nucleotides in length.

The percent complementarity between the targeting sequence (guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some cases, the percent complementarity between the targeting sequence of the targeting segment and the target site of the target nucleic acid is 100% over the seven contiguous 5′-most nucleotides of the target site of the target nucleic acid. In some cases, the percent complementarity between the targeting sequence of the targeting segment and the target site of the target nucleic acid is 60% or more over about 20 contiguous nucleotides. In some cases, the percent complementarity between the targeting sequence of the targeting segment and the target site of the target nucleic acid is 100% over the fourteen contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the seven contiguous 5′-most nucleotides of the target site of the target nucleic acid and as low as 0% or more over the remainder. In such a case, the targeting sequence can be considered to be 20 nucleotides in length.

In some cases, the percent complementarity between the targeting sequence of the targeting segment and the target site of the target nucleic acid is 100% over the 7 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 8 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 9 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 10 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 17 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 18 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over about 20 contiguous nucleotides.

In some cases, the percent complementarity between the targeting sequence of the targeting segment and the target site of the target nucleic acid is 100% over the 7 contiguous 5′-most nucleotides of the target site of the target nucleic acid and as low as 0% or more over the remainder. In such a case, the targeting sequence can be considered to be 7 nucleotides in length. In some cases, the percent complementarity between the targeting sequence of the targeting segment and the target site of the target nucleic acid is 100% over the 8 contiguous 5′-most nucleotides of the target site of the target nucleic acid and as low as 0% or more over the remainder. In such a case, the targeting sequence can be considered to be 8 nucleotides in length. In some cases, the percent complementarity between the targeting sequence of the targeting segment and the target site of the target nucleic acid is 100% over the 9 contiguous 5′-most nucleotides of the target site of the target nucleic acid and as low as 0% or more over the remainder. In such a case, the targeting sequence can be considered to be 9 nucleotides in length. In some cases, the percent complementarity between the targeting sequence of the targeting segment and the target site of the target nucleic acid is 100% over the 10 contiguous 5′-most nucleotides of the target site of the target nucleic acid and as low as 0% or more over the remainder. In such a case, the targeting sequence can be considered to be 10 nucleotides in length. In some cases, the percent complementarity between the targeting sequence of the targeting segment and the target site of the target nucleic acid is 100% over the 11 contiguous 5′-most nucleotides of the target site of the target nucleic acid and as low as 0% or more over the remainder. In such a case, the targeting sequence can be considered to be 11 nucleotides in length. In some cases, the percent complementarity between the targeting sequence of the targeting segment and the target site of the target nucleic acid is 100% over the 12 contiguous 5′-most nucleotides of the target site of the target nucleic acid and as low as 0% or more over the remainder. In such a case, the targeting sequence can be considered to be 12 nucleotides in length. In some cases, the percent complementarity between the targeting sequence of the targeting segment and the target site of the target nucleic acid is 100% over the 13 contiguous 5′-most nucleotides of the target site of the target nucleic acid and as low as 0% or more over the remainder. In such a case, the targeting sequence can be considered to be 13 nucleotides in length. In some cases, the percent complementarity between the targeting sequence of the targeting segment and the target site of the target nucleic acid is 100% over the 14 contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the 17 contiguous 5′-most nucleotides of the target site of the target nucleic acid and as low as 0% or more over the remainder. In such a case, the targeting sequence can be considered to be 17 nucleotides in length. In some cases, the percent complementarity between the targeting sequence of the targeting segment and the target site of the target nucleic acid is 100% over the 18 contiguous 5′-most nucleotides of the target site of the target nucleic acid and as low as 0% or more over the remainder. In such a case, the targeting sequence can be considered to be 18 nucleotides in length.

### Protein-Binding Segment of a Cas9 Guide RNA

The protein-binding segment of a subject Cas9 guide RNA interacts with a Cas9 protein. The Cas9 guide RNA guides the bound Cas9 protein to a specific nucleotide sequence within target nucleic acid via the above mentioned targeting segment. The protein-binding segment of a Cas9 guide RNA comprises two stretches of nucleotides that are complementary to one another and hybridize to form a double stranded RNA duplex (dsRNA duplex). Thus, the protein-binding segment includes a dsRNA duplex. In some cases, the protein-binding segment also includes stem loop 1 (the “nexus”) of a Cas9 guide RNA. For example, in some cases, the activator of a Cas9 guide RNA (dgRNA or sgRNA) includes (i) a duplex forming segment that contributes to the dsRNA duplex of the protein-binding segment; and (ii) nucleotides 3′ of the duplex forming segment, e.g., that form stem loop 1 (the “nexus”). For example, in some cases, the protein-binding segment includes stem loop 1 (the “nexus”) of a Cas9 guide RNA. In some cases, the protein-binding segment includes 5 or more nucleotides (nt) (e.g., 6 or more, 7 or more, 8 or more, 9 or more, 10 or more, 11 or more, 12 or more, 15 or more, 20 or more, 30 or more, 40 or more, 50 or more, 60 or more, 70 or more, 75 or more, or 80 or more nt) 3′ of the dsRNA duplex (where 3′ is relative to the duplex-forming segment of the activator sequence).

The dsRNA duplex of the guide RNA (sgRNA or dgRNA) that forms between the activator and targeter is sometimes referred to herein as the “stem loop”. In addition, the activator (activator RNA, tracrRNA) of many naturally existing Cas9 guide RNAs (e.g., S. pygogenes guide RNAs) has 3 stem loops (3 hairpins) that are 3′ of the duplex-forming segment of the activator. The closest stem loop to the duplex-forming segment of the activator (3′ of the duplex forming segment) is called “stem loop 1” (and is also referred to herein as the “nexus”); the next stem loop is called “stem loop 2” (and is also referred to herein as the “hairpin 1”); and the next stem loop is called “stem loop 3” (and is also referred to herein as the “hairpin 2”).

In some cases, a Cas9 guide RNA (sgRNA or dgRNA) (e.g., a full length Cas9 guide RNA) has stem loops 1, 2, and 3. In some cases, an activator (of a Cas9 guide RNA) has stem loop 1, but does not have stem loop 2 and does not have stem loop 3. In some cases, an activator (of a Cas9 guide RNA) has stem loop 1 and stem loop 2, but does not have stem loop 3. In some cases, an activator (of a Cas9 guide RNA) has stem loops 1, 2, and 3.

In some cases, the activator (e.g., tracr sequence) of a Cas9 guide RNA (dgRNA or sgRNA) includes (i) a duplex forming segment that contributes to the dsRNA duplex of the protein-binding segment; and (ii) a stretch of nucleotides (e.g., referred to herein as a 3′ tail) 3′ of the duplex forming segment. In some cases, the additional nucleotides 3′ of the duplex forming segment form stem loop 1. In some cases, the activator (e.g., tracr sequence) of a Cas9 guide RNA (dgRNA or sgRNA) includes (i) a duplex forming segment that contributes to the dsRNA duplex of the protein-binding segment; and (ii) 5 or more nucleotides (e.g., 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, 50 or more, 60 or more, 70 or more, or 75 or more nucleotides) 3′ of the duplex forming segment. In some cases, the activator (activator RNA) of a Cas9 guide RNA (dgRNA or sgRNA) includes (i) a duplex forming segment that contributes to the dsRNA duplex of the protein-binding segment; and (ii) 5 or more nucleotides (e.g., 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, 50 or more, 60 or more, 70 or more, or 75 or more nucleotides) 3′ of the duplex forming segment.

In some cases, the activator (e.g., tracr sequence) of a Cas9 guide RNA (dgRNA or sgRNA) includes (i) a duplex forming segment that contributes to the dsRNA duplex of the protein-binding segment; and (ii) a stretch of nucleotides (e.g., referred to herein as a 3′ tail) 3′ of the duplex forming segment. In some cases, the stretch of nucleotides 3′ of the duplex forming segment has a length in a range of from 5 to 200 nucleotides (nt) (e.g., from 5 to 150 nt, from 5 to 130 nt, from 5 to 120 nt, from 5 to 100 nt, from 5 to 80 nt, from 10 to 200 nt, from 10 to 150 nt, from 10 to 130 nt, from 10 to 120 nt, from 10 to 100 nt, from 10 to 80 nt, from 12 to 200 nt, from 12 to 150 nt, from 12 to 130 nt, from 12 to 120 nt, from 12 to 100 nt, from 12 to 80 nt, from 15 to 200 nt, from 15 to 150 nt, from 15 to 130 nt, from 15 to 120 nt, from 15 to 100 nt, from 15 to 80 nt, from 20 to 200 nt, from 20 to 150 nt, from 20 to 130 nt, from 20 to 120 nt, from 20 to 100 nt, from 20 to 80 nt, from 30 to 200 nt, from 30 to 150 nt, from 30 to 130 nt, from 30 to 120 nt, from 30 to 100 nt, or from 30 to 80 nt). In some cases, the nucleotides of the 3′ tail of an activator RNA are wild type sequences. Although a number of different alternative sequences can be used, an example Cas9 single guide RNA (based on crRNA and tracrRNA from S. pyogenes, where the dsRNA duplex of the protein-binding segment is truncated relative to the dsRNA duplex present in the wild type dual guide RNA) can include the sequence set forth in SEQ ID NO: 958 (This example sequence does not include the guide sequence. The guide sequence, which varies depending on the target, would be 5′ of this example sequence. The activator in this example is 66 nucleotides long).

Examples of various Cas9 proteins and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

### Guide RNAs Corresponding to Type V and Type VI CRISPR/Cas Endonucleases (e.g., Cpf1 Guide RNA)

A guide RNA that binds to a type V or type VI CRISPR/Cas protein (e.g., Cpf1, C2c1, C2c2, C2c3), and targets the complex to a specific location within a target nucleic acid is referred to herein generally as a “type V or type VI CRISPR/Cas guide RNA”. An example of a more specific term is a “Cpf1 guide RNA.”

A type V or type VI CRISPR/Cas guide RNA (e.g., cpf1 guide RNA) can have a total length of from 30 nucleotides (nt) to 200 nt, e.g., from 30 nt to 180 nt, from 30 nt to 160 nt, from 30 nt to 150 nt, from 30 nt to 125 nt, from 30 nt to 100 nt, from 30 nt to 90 nt, from 30 nt to 80 nt, from 30 nt to 70 nt, from 30 nt to 60 nt, from 30 nt to 50 nt, from 50 nt to 200 nt, from 50 nt to 180 nt, from 50 nt to 160 nt, from 50 nt to 150 nt, from 50 nt to 125 nt, from 50 nt to 100 nt, from 50 nt to 90 nt, from 50 nt to 80 nt, from 50 nt to 70 nt, from 50 nt to 60 nt, from 70 nt to 200 nt, from 70 nt to 180 nt, from 70 nt to 160 nt, from 70 nt to 150 nt, from 70 nt to 125 nt, from 70 nt to 100 nt, from 70 nt to 90 nt, or from 70 nt to 80 nt). In some cases, a type V or type VI CRISPR/Cas guide RNA (e.g., cpf1 guide RNA) has a total length of at least 30 nt (e.g., at least 40 nt, at least 50 nt, at least 60 nt, at least 70 nt, at least 80 nt, at least 90 nt, at least 100 nt, or at least 120 nt,).

In some cases, a Cpf1 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt.

Like a Cas9 guide RNA, a type V or type VI CRISPR/Cas guide RNA (e.g., cpf1 guide RNA) can include a target nucleic acid-binding segment and a duplex-forming region (e.g., in some cases formed from two duplex-forming segments, i.e., two stretches of nucleotides that hybridize to one another to form a duplex).

The target nucleic acid-binding segment of a type V or type VI CRISPR/Cas guide RNA (e.g., cpf1 guide RNA) can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some cases, the target nucleic acid-binding segment has a length of 23 nt. In some cases, the target nucleic acid-binding segment has a length of 24 nt. In some cases, the target nucleic acid-binding segment has a length of 25 nt.

The guide sequence of a type V or type VI CRISPR/Cas guide RNA (e.g., cpf1 guide RNA) can have a length of from 15 nt to 30 nt (e.g., 15 to 25 nt, 15 to 24 nt, 15 to 23 nt, 15 to 22 nt, 15 to 21 nt, 15 to 20 nt, 15 to 19 nt, 15 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt.

The guide sequence of a type V or type VI CRISPR/Cas guide RNA (e.g., cpf1 guide RNA) can have 100% complementarity with a corresponding length of target nucleic acid sequence. The guide sequence can have less than 100% complementarity with a corresponding length of target nucleic acid sequence. For example, the guide sequence of a type V or type VI CRISPR/Cas guide RNA (e.g., cpf1 guide RNA) can have 1, 2, 3, 4, or 5 nucleotides that are not complementary to the target nucleic acid sequence. For example, in some cases, where a guide sequence has a length of 25 nucleotides, and the target nucleic acid sequence has a length of 25 nucleotides, in some cases, the target nucleic acid-binding segment has 100% complementarity to the target nucleic acid sequence. As another example, in some cases, where a guide sequence has a length of 25 nucleotides, and the target nucleic acid sequence has a length of 25 nucleotides, in some cases, the target nucleic acid-binding segment has 1 non-complementary nucleotide and 24 complementary nucleotides with the target nucleic acid sequence. As another example, in some cases, where a guide sequence has a length of 25 nucleotides, and the target nucleic acid sequence has a length of 25 nucleotides, in some cases, the target nucleic acid-binding segment has 2 non-complementary nucleotides and 23 complementary nucleotides with the target nucleic acid sequence.

The duplex-forming segment of a type V or type VI CRISPR/Cas guide RNA (e.g., cpf1 guide RNA) (e.g., of a targeter RNA or an activator RNA) can have a length of from 15 nt to 25 nt (e.g., 15 nt, 16 nt, 17 nt, 18 nt, 19 nt, 20 nt, 21 nt, 22 nt, 23 nt, 24 nt, or 25 nt).

The RNA duplex of a type V or type VI CRISPR/Cas guide RNA (e.g., cpf1 guide RNA) can have a length of from 5 base pairs (bp) to 40 bp (e.g., from 5 to 35 bp, 5 to 30 bp, 5 to 25 bp, 5 to 20 bp, 5 to 15 bp, 5-12 bp, 5-10 bp, 5-8 bp, 6 to 40 bp, 6 to 35 bp, 6 to 30 bp, 6 to 25 bp, 6 to 20 bp, 6 to 15 bp, 6 to 12 bp, 6 to 10 bp, 6 to 8 bp, 7 to 40 bp, 7 to 35 bp, 7 to 30 bp, 7 to 25 bp, 7 to 20 bp, 7 to 15 bp, 7 to 12 bp, 7 to 10 bp, 8 to 40 bp, 8 to 35 bp, 8 to 30 bp, 8 to 25 bp, 8 to 20 bp, 8 to 15 bp, 8 to 12 bp, 8 to 10 bp, 9 to 40 bp, 9 to 35 bp, 9 to 30 bp, 9 to 25 bp, 9 to 20 bp, 9 to 15 bp, 9 to 12 bp, 9 to 10 bp, 10 to 40 bp, 10 to 35 bp, 10 to 30 bp, 10 to 25 bp, 10 to 20 bp, 10 to 15 bp, or 10 to 12 bp).

As an example, a duplex-forming segment of a Cpf1 guide RNA can comprise a nucleotide sequence selected from (5′ to 3′): AAUUUCUACUGUUGUAGAU (SEQ ID NO: 1093), AAUUUCUGCUGUUGCAGAU (SEQ ID NO: 1094), AAUUUCCACUGUUGUGGAU (SEQ ID NO: 1095), AAUUCCUACUGUUGUAGGU (SEQ ID NO: 1096), AAUUUCUACUAUUGUAGAU (SEQ ID NO: 1097), AAUUUCUACUGCUGUAGAU (SEQ ID NO: 1098), AAUUUCUACUUUGUAGAU (SEQ ID NO: 1099), and AAUUUCUACUUGUAGAU (SEQ ID NO: 1100). The guide sequence can then follow (5′ to 3′) the duplex forming segment.

A non-limiting example of an activator RNA (e.g. tracrRNA) of a C2c1 guide RNA (dual guide or single guide) is an RNA that includes the nucleotide sequence GAAUUUUUCAACGGGUGUGCCAAUGGCCACUUUCCAGGUGGCAAAGCCCGUUGA GCUUCUCAAAAAG (SEQ ID NO: 1101). In some cases, a C2c1 guide RNA (dual guide or single guide) is an RNA that includes the nucleotide sequence In some cases, a C2c1 guide RNA (dual guide or single guide) is an RNA that includes the nucleotide sequence GUCUAGAGGACAGAAUUUUUCAACGGGUGUGCCAAUGGCCACUUUCCAGGUGGC AAAGCCCGUUGAGCUUCUCAAAAAG (SEQ ID NO: 1102). In some cases, a C2c1 guide RNA (dual guide or single guide) is an RNA that includes the nucleotide sequence UCUAGAGGACAGAAUUUUUCAACGGGUGUGCCAAUGGCCACUUUCCAGGUGGCA AAGCCCGUUGAGCUUCUCAAAAAG (SEQ ID NO: 1103). A non-limiting example of an activator RNA (e.g. tracrRNA) of a C2c1 guide RNA (dual guide or single guide) is an RNA that includes the nucleotide sequence ACUUUCCAGGCAAAGCCCGUUGAGCUUCUCAAAAAG (SEQ ID NO: 1104). In some cases, a duplex forming segment of a C2c1 guide RNA (dual guide or single guide) of an activator RNA (e.g. tracrRNA) includes the nucleotide sequence AGCUUCUCA (SEQ ID NO: 1105) or the nucleotide sequence GCUUCUCA (SEQ ID NO: 1106) (the duplex forming segment from a naturally existing tracrRNA.

A non-limiting example of a targeter RNA (e.g. crRNA) of a C2c1 guide RNA (dual guide or single guide) is an RNA with the nucleotide sequence CUGAGAAGUGGCACNNNNNNNNNNNNNNNNNNNN (SEQ ID NO: 1107), where the Ns represent the guide sequence, which will vary depending on the target sequence, and although 20 Ns are depicted a range of different lengths are acceptable. In some cases, a duplex forming segment of a C2c1 guide RNA (dual guide or single guide) of a targeter RNA (e.g. crRNA) includes the nucleotide sequence CUGAGAAGUGGCAC (SEQ ID NO: 1108) or includes the nucleotide sequence CUGAGAAGU (SEQ ID NO: 1109) or includes the nucleotide sequence UGAGAAGUGGCAC (SEQ ID NO: 1110) or includes the nucleotide sequence UGAGAAGU (SEQ ID NO: 1111).

Examples and guidance related to type V or type VI CRISPR/Cas endonucleases and guide RNAs (as well as information regarding requirements related to protospacer adjacent motif (PAM) sequences present in targeted nucleic acids) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97.

### Target Genomic DNA

A target nucleic acid (e.g., target genomic DNA) is located within a zygote.

A target genomic DNA can be any genomic DNA in which the sequence is to be modified, e.g., by substitution and/or insertion and/or deletion of one or more nucleotides present in the target genomic DNA.

Target genes (target genomic DNA) include those genes involved in various diseases or conditions. In some cases, the target genomic DNA is mutated, such that it encodes a non-functional polypeptide, or such that a polypeptide encoded by the target genomic DNA is not synthesized in any detectable amount, or such that a polypeptide encoded by the target genomic DNA is synthesized in a lower than normal amount, such that an individual having the mutation has a disease. Such diseases include, but are not limited to, achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, Crigler-Najjer Syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), Glycogen Storage Disease Type IV, hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome. Other such diseases include, e.g., acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

For example, in some cases, the target genomic DNA comprises a mutation that gives rise to a trinucleotide repeat disease. Exemplary trinucleotide repeat diseases and target genes involved in trinucleotide repeat diseases Trinucleotide Repeat Diseases Gene DRPLA (Dentatorubropallidoluysian atrophy) ATN1 or DRPLA HD (Huntington's disease) HTT (Huntingtin) SBMA (Spinobulbar muscular atrophy or Androgen receptor on the Kennedy disease) X chromosome. SCA1 (Spinocerebellar ataxia Type 1) ATXN1 SCA2 (Spinocerebellar ataxia Type 2) ATXN2 SCA3 (Spinocerebellar ataxia Type 3 or ATXN3 Machado-Joseph disease) SCA6 (Spinocerebellar ataxia Type 6) CACNA1A SCA7 (Spinocerebellar ataxia Type 7) ATXN7 SCA17 (Spinocerebellar ataxia Type 17) TBP FRAXA (Fragile X syndrome) FMR1, on the X-chromosome FXTAS (Fragile X-associated tremor/FMR1, on the X-ataxia syndrome) chromosome FRAXE (Fragile XE mental retardation) AFF2 or FMR2, on the X-chromosome FRDA (Friedreich's ataxia) FXN or X25, (frataxin-reduced expression) DM (Myotonic dystrophy) DMPK SCA8 (Spinocerebellar ataxia Type 8) OSCA or SCA8 SCA12 (Spinocerebellar ataxia Type 12) PPP2R2B or SCA12.

For example, in some cases, a suitable target genomic DNA is a β-globin gene, e.g., a β-globin gene with a sickle cell mutation. As another example, a suitable target genomic DNA is a Huntington's locus, e.g., an HIT gene, where the HTT gene comprises a mutation (e.g., a CAG repeat expansion comprising more than 35 CAG repeats) that gives rise to Huntington's Disease. As another example, a suitable target genomic DNA is an adenosine deaminase gene that comprises a mutation that gives rise to severe combined immunodeficiency. As another example, a suitable target genomic DNA is a BCL11A gene comprising a mutation associated with control of the gamma-globin genes.

### Donor Polynucleotide

In some cases, a genome targeting composition comprises a donor template nucleic acid (“donor polynucleotide”). In some cases, a method of the present disclosure comprises contacting the target DNA with a donor polynucleotide, wherein the donor polynucleotide, a portion of the donor polynucleotide, a copy of the donor polynucleotide, or a portion of a copy of the donor polynucleotide integrates into the target DNA (e.g., via homology-directed repair). In some cases, the method does not comprise contacting the cell with a donor polynucleotide (e.g., resulting in non-homologous end-joining). A donor poly nucleotide can be introduced into a target cell using any convenient technique for introducing nucleic acids into cells.

When it is desirable to insert a polynucleotide sequence into a target DNA sequence, a polynucleotide comprising a donor sequence to be inserted is provided to the cell (e.g., the target DNA is contacted with a donor polynucleotide in addition to a genome targeting composition (e.g., a genome editing endonuclease; or a genome-editing endonuclease and a guide RNA). By a “donor sequence” or “donor polynucleotide” it is meant a nucleic acid sequence to be inserted at the cleavage site induced by a genome-editing endonuclease. A suitable donor polynucleotide can be single stranded or double stranded. For example, in some cases, a donor polynucleotide is single stranded (e.g., in some cases can be referred to as an oligonucleotide), and in some cases a donor polynucleotide is double stranded (e.g., in some cases can be include two separate oligonucleotides that are hybridized). The donor polynucleotide will contain sufficient homology to a genomic sequence at the cleavage site, e.g. 70%, 80%, 85%, 90%, 95%, or 100% homology with the nucleotide sequences flanking the cleavage site, e.g. within 100 bases or less (e.g., 50 bases or less of the cleavage site, e.g. within 30 bases, within 15 bases, within 10 bases, within 5 bases, or immediately flanking the cleavage site), to support homology-directed repair between it and the genomic sequence to which it bears homology. Approximately 25 nucleotides (nt) or more (e.g., 30 nt or more, 40 nt or more, 50 nt or more, 60 nt or more, 70 nt or more, 80 nt or more, 90 nt or more, 100 nt or more, 150 nt or more, 200 nt or more, etc.) of sequence homology between a donor and a genomic sequence (or any integral value between 10 and 200 nucleotides, or more) can support homology-directed repair. For example, in some cases, the 5′ and/or the 3′ flanking homology arm (e.g., in some cases both of the flanking homology arms) of a donor polynucleotide can be 30 nucleotides (nt) or more in length (e.g., 40 nt or more, 50 nt or more, 60 nt or more, 70 nt or more, 80 nt or more, 90 nt or more, 100 nt or more, etc.). For example, in some cases, the 5′ and/or the 3′ flanking homology arm (e.g., in some cases both of the flanking homology arms) of a donor polynucleotide can have a length in a range of from 30 nt to 500 nt (e.g., 30 nt to 400 nt, 30 nt to 350 nt, 30 nt to 300 nt, 30 nt to 250 nt, 30 nt to 200 nt, 30 nt to 150 nt, 30 nt to 100 nt, 30 nt to 90 nt, 30 nt to 80 nt, 50 nt to 400 nt, 50 nt to 350 nt, 50 nt to 300 nt, 50 nt to 250 nt, 50 nt to 200 nt, 50 nt to 150 nt, 50 nt to 100 nt, 50 nt to 90 nt, 50 nt to 80 nt, 60 nt to 400 nt, 60 nt to 350 nt, 60 nt to 300 nt, 60 nt to 250 nt, 60 nt to 200 nt, 60 nt to 150 nt, 60 nt to 100 nt, 60 nt to 90 nt, 60 nt to 80 nt).

Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.”

In some cases, a donor polynucleotide is delivered to the zygote (introduced into a zygote) as part of recombinant viral vector (e.g., an adeno-associated virus (AAV) vector; a lentiviral vector; etc.). For example a recombinant viral DNA vector can include a donor polynucleotide sequence (donor sequence) (e.g., a recombinant viral DNA vector can include a DNA molecule that includes a donor polynucleotide sequence). In some cases, a donor polynucleotide is introduced into a zygote as a recombinant viral DNA vector (e.g., the donor polynucleotide sequence is present as part of the viral DNA) and the genome-editing endonuclease (e.g., Cas9 protein; etc.) and, where applicable, a guide RNA are delivered by a different route. For example, in some cases, a donor polynucleotide is introduced into a zygote as a recombinant virus vector (e.g., the donor polynucleotide sequence is present as part of the recombinant viral vector and a Cas9 protein and Cas9 guide RNA are delivered as part of a separate expression vector. In some cases, a donor polynucleotide is introduced into a zygote as a recombinant viral vector; (e.g., the donor polynucleotide sequence is present as part of the recombinant viral vector) and a Cas9 protein and Cas9 guide RNA are delivered as part of a ribonucleoprotein complex (RNP). In some cases: (i) a donor polynucleotide is introduced into a zygote as a recombinant viral vector (e.g., the donor polynucleotide sequence is present as part of the recombinant viral vector), (ii) a Cas9 guide RNA is delivered as either an RNA or DNA encoding the RNA, and (iii) a Cas9 protein is delivered as a protein or as a nucleic acid encoding the protein (e.g., RNA or DNA).

In some cases, a recombinant viral vector (e.g., a recombinant AAV vector, a recombinant lentiviral vector, a recombinant retroviral vector; etc.) comprising a donor polynucleotide is introduced into a zygote before a Cas9-guide RNA RNP is introduced into the cell. For example, in some cases, a recombinant viral vector comprising a donor polynucleotide is introduced into a zygote from 2 hours to 72 hours (e.g., from 2 hours to 4 hours, from 4 hours to 8 hours, from 8 hours to 12 hours, from 12 hours to 24 hours, from 24 hours to 48 hours, or from 48 hours to 72 hours) before the Cas9-guide RNA RNP is introduced into the zygote.

Introducing a Genome-Modifying Composition into a Zygote

A genome-modifying composition can be introduced into a zygote by electroporation. An electroporation mixture, comprising: a) a genome-modifying composition; and b) one zygote or a plurality of zygotes. Suitable genome-modifying compositions are described above. A genome-modifying composition can comprise an RNP comprising: i) an RNA-guided endonuclease (e.g., a CRISPR/Cas polypeptide); and ii) one or more guide RNAs. A genome-modifying composition can comprise an RNP comprising: i) an RNA-guided endonuclease (e.g., a CRISPR/Cas polypeptide); ii) one or more guide RNAs; and iii) a donor template DNA. A genome-modifying composition can comprise: a) an RNP comprising: i) an RNA-guided endonuclease (e.g., a CRISPR/Cas polypeptide); and ii) one or more guide RNAs; and b) a donor template DNA.

A method of the present disclosure involves electroporating a ribonucleoprotein (RNP) complex into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a genome targeting composition, forming a zygote/genome targeting composition; and b) electroporating the zygote/genome targeting composition with 2 pulses at 30 V, where each pulse is a 3-millisecond (msec) pulse, with a 1 msec interval between the 2 pulses. In some cases, at least 50%, at least 60%, at least 70%, at least 80%, at least 85%, at least 90%, at least 95%, or at least 98%, of the zygotes are viable after electroporation. In some cases, from 50% to 95% of the zygotes are viable after electroporation. In some cases, from 60% to 95% of the zygotes are viable after electroporation. In some cases, from 70% to 95% of the zygotes are viable after electroporation. In some cases, from 80% to 95% of the zygotes are viable after electroporation. In some cases, 100% of the zygotes are viable after electroporation. In some cases, the genomic modification occurs via HDR or NHEJ. In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

A method of the present disclosure involves electroporating a ribonucleoprotein (RNP) complex into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a genome targeting composition, forming a zygote/genome targeting composition; and b) electroporating the zygote/genome targeting composition with 6 pulses at 30 V per pulse, where each pulse is a 3-millisecond (msec) pulse, with a 1 msec interval between consecutive pulses. In some cases, at least 50%, at least 60%, at least 70%, at least 80%, at least 85%, at least 90%, at least 95%, or at least 98%, of the zygotes are viable after electroporation. In some cases, from 50% to 95% of the zygotes are viable after electroporation. In some cases, from 60% to 95% of the zygotes are viable after electroporation. In some cases, from 70% to 95% of the zygotes are viable after electroporation. In some cases, from 80% to 95% of the zygotes are viable after electroporation. In some cases, 100% of the zygotes are viable after electroporation. In some cases, the genomic modification occurs via HDR or NHEJ. In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

A method of the present disclosure involves electroporating a ribonucleoprotein (RNP) complex into a zygote. In some cases, a method of the present disclosure comprises: a) combining, in an electroporation container (e.g., an electroporation cuvette) a zygote or a plurality of zygotes (e.g., from 1 zygote to 150 zygotes; e.g., from 1 zygote to 5 zygotes, from 10 zygotes to 15 zygotes, from 15 zygotes to 20 zygotes, from 20 zygotes to 25 zygotes, from 25 zygotes to 30 zygotes, from 30 zygotes to 35 zygotes, from 35 zygotes to 40 zygotes, from 40 zygotes to 45 zygotes, from 45 zygotes to 50 zygotes, from 50 zygotes to 75 zygotes, from 75 zygotes to 100 zygotes, from 100 zygotes to 120 zygotes, from 120 zygotes to 140 zygotes, or from 140 zygotes to 150 zygotes) in a suitable liquid medium with an equal volume of a genome targeting composition, forming a zygote/genome targeting composition; and b) electroporating the zygote/genome targeting composition with a single pulse at 30 V, where the single pulse is a 3-millisecond (msec) pulse. In some cases, the RNP is present in the electroporation composition at a concentration of from 5 μM to 16 μM. In some cases, the RNP is present in the electroporation composition at a concentration of 8 μM. In some cases, at least 50%, at least 60%, at least 70%, at least 80%, at least 85%, at least 90%, at least 95%, or at least 98%, of the zygotes are viable after electroporation with the RNP. In some cases, from 50% to 95% of the zygotes are viable after electroporation with the RNP. In some cases, from 60% to 95% of the zygotes are viable after electroporation with the RNP. In some cases, from 70% to 95% of the zygotes are viable after electroporation with the RNP. In some cases, from 80% to 95% of the zygotes are viable after electroporation with the RNP. In some cases, 100% of the zygotes are viable after electroporation with the RNP. In some cases, the genomic modification occurs via homology-directed repair (HDR) or non-homologous end joining (NHEJ). In some cases, the genomic modification occurs via HDR, and wherein the efficiency of HDR is at least 50%, at least 60%, at least 70%, at least 80%, at least 90%, or 100%. In some cases, the genomic modification occurs via NHEJ, and wherein the efficiency of NHEJ is at least 50%, at least 60%, at least 70%, at least 80%, at least 90%, or 100%.

In some cases, the RNP complex comprises an RNA and a DNA-binding polypeptide, where the RNA and the DNA-binding polypeptide are present in a ratio of from 0.5:1 to 1:1, from 1:1 to 1:1.5, or from 1:1.5 to 1:2 RNA:DNA-binding polypeptide. In some cases, the RNP complex is present in the electroporation mixture at a concentration of from 5 μM to 15 μM, e.g., from 5 μM to 10 μM, or from 10 μM to 15 μM. In some cases, the RNP complex is present in the electroporation mixture at a concentration of 8 μM. In some cases, the electroporation mixture includes a donor DNA template. The donor DNA template can be part of the RNP, or can be separate from the RNP.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Genome Editing in Zygotes Via NHEJ or HDR

A method to directly deliver Cas9:sgRNA ribonucleoproteins (RNPs) into mouse zygotes by electroporation, using standard commercially available equipment and reagents common to most biological labs, is described. This method is called CRISPR RNP electroporation of zygotes (CRISPR-EZ). The use of CRISPR-EZ leads to genome editing in zygotes and generates animals with homogeneous genetic modifications.

Using a sgRNA targeting tyrosinase (tyr), a key enzyme for pigment synthesis, live animals were generated with 100% editing efficiency (NHEJ or HDR), of which 88% exhibiting bi-allelic editing and 42% harboring a HDR-mediated modification. CRISPR-EZ edited embryos exhibited a significant increase in survival; and edited animals were viable and germline competent. This CRISPR-EZ technology has been employed for genome editing on multiple genes, and high efficiency editing was consistently obtained in generating a variety of desired genomic modifications, including indel mutations, precise deletion and small precise insertions. Taken together, CRISPR-EZ is a simple, economic, high-throughput, and highly efficient technique for genome editing in vivo, which has a great potential to replace the traditional microinjection-dependent technique in a variety of mammalian species.

### Materials and Methods

Designing Single Guide RNA (sgRNA)

Using one of many online resources (e.g., http:(double forward slash)crispr(dot)dfci(dot)harvard(dot)edu/SSC/), input target DNA sequence. The precise choice of sgRNA(s) largely depends on the needs of the researcher. Inserts, deletions and Knock-Ins all have different criteria for selection of sgRNA. Choose three or four sgRNAs with reasonably high scores (e.g., 0.80 or higher).

In Vitro T7 Transcription of sgRNA

For each sgRNA, a synthetically assembled oligonucleotide (Integrated DNA technologies, San Diego, Calif.) DNA Template is generated by overlapping polymerase chain reaction (PCR) that includes a T7 Promoter followed by the 20 nt target sequence obtained from previous section, and concluded with 15 nt that hybridize to an optimized sgRNA scaffold.

Briefly, for each sgRNA template, the 50 μL PCR reaction included 0.02 M uniquely designed oligonucleotide (5′-GGA TCC TAA TAC GAC TCA CTA TAG—guide-sequence—GTT TTA GAG CTA GAA), while the remaining reagents are common to all template synthesis reactions; 0.02 μM T7RevLong (5′AAA AAA GCA CCG ACT CGG TGC CAC TTT TTC AAG TTG ATA ACG GAC TAG CCT TAT TTT AAC TTG CTA TTT CTA GCT CTA AAA C) (SEQ ID NO:1143), 1 μM T7FwdAmp (5′-GGA TCC TAA TAC GAC TCA CTA TAG) (SEQ ID NO:1144). 1 μM T7RevAmp (5′-AAA AAA GCA CCG ACT CGG) (SEQ ID NO: 1145), 10 mM dNTPs and Phusion Polymerase (NEB m0530, Ipswich, Mass.) according to manufacturer's protocol. The thermocycler setting consisted of 30 cycles of 95° C. for 10 s, 57° C. for 10 s and 72° C. for 10 s. Following the PCR reaction, the product may be frozen at −20° C. or used immediately. The sequences for all of the sgRNAs used in this project were: sgTyr (5′ GGG TGG ATG ACC GTG AGT CC) (SEQ ID NO:1146), sgCdh1 (5′TAT GAC TGG AGT CCC GGG CG) (SEQ ID NO:1147), sgCdk8 (5′AGA CAG AAA CAC CTT CAG AA) (SEQ ID NO:1148), sgKif11 (5′CGT GGA ATT ATA CCA GCC AG) (SEQ ID NO:1149), Mecp2 R1 (5′AGG AGT GAG GTC TAG TAC TT) (SEQ ID NO:1150), Mecp2 L2 (5′ CCC AAG GAT ACA GTA TCC TA) (SEQ ID NO:1151).

The 20 uL In Vitro Transcription (IVT) reaction consists of 25 ng/μL of PCR amplified DNA template, 10 mM nucleotide triphosphates (NTPs) and T7 RNA Polymerase enzyme and reaction buffer (NEB E2040S) as per manufacturer's protocol. The reaction is mixed by gentle pipetting and placed in a thermocycler set to 37° C. for more than 18 hrs. At the end of the incubation period, 1 μL of RNAse-Free DNASE (NEB M0303S) is added and further incubated at room temperature (RT=22-25° C.).

To purify the IVT reaction, the total volume is brought to 150 uL with 100% Ethanol. To this, 100 μL of 5× AmpureXL (Beckman Coulter A63880, or equivalent reagent, such as MagNa beads as described in Rohland 2012) for solid-phase reversible immobilization (SPRI) for RNA cleanup. The reaction is mixed by pipetting ten times and left to incubate at room temperature (RT) for five minutes. Reactions are placed on a magnetic stand (Invitrogen 12321D) for 5 minutes, until pellet is formed. Supernatant is carefully discarded, so as to not disturb newly formed pellet. To wash the pellet, 80% Ethanol is pipetted gently over the pellet and allowed to sit for 2 minutes. This was then repeated for a total of two wash steps. The supernatant is again carefully discarded and the pellet is allowed to air dry for ten minutes. To elute the RNA, the reaction is removed from the magnetic stand and pellet is pipetted with 20 μL of RNASE-Free H₂O (AMBION AM9937) and allowed to incubate at RT for two minutes. The reaction was then placed back onto the stand for an additional five minutes at which point the supernatant is carefully transferred to a RNASE-Free tube (VWR 211-0319) for storage in −80° C.

### Intraperitoneal Injection of Superovulation Hormones

Female Mice (C57BL/6J JAX 000664), aged 3-5 weeks, are collected. Superovulation of the female mice is initiated via intraperitoneal injection (IP) of approximately 5 IU (international Units) of Pregnant Mare Serum Gonadotropin (PMSG) (Calbiochem, Millipore: Cat#367222), followed by injection of Human Chorion Gonadotropin (HCG) (Calbiochem (Millipore: Cat#230734) administered 46-48 hrs after PMSG. Lyophilized (1 mg=1000 IU) PMSG stock is reconstituted in 20 mL of bacteriostatic sterile saline (CATALOG), and Lyophilized (1 mg=3000 IU) HCG stock is reconstituted in 60 mL of bacteriostatic sterile saline to obtain the working stock concentrations of 50 IU/mL. Both hormone stocks are maintained in aliquots of 600 μL at −80° C. until the time of injection, at which time the aliquot is thawed to room temperature immediately prior to the IP injection. For IP injections, 100 μL of PMSG (and then HCG) stock solution is administered, typically between 1-2 pm on Day 1, which introduces 5 IU of PMSG into the female mouse. Immediately after HCG injection, females are housed 1:1 with 3-8 month old males of proven fertility.

### Embryo Harvest

The morning after HcG IP injection, females are checked for the presence of a copulation plug. The plugged mice are sacrificed by asphyxiation (CO₂) followed by cervical dislocation. Pronucleus stage embryos of approximately 0.5 days post coitum (0.5 dpc) are collected by surgically opening abdominal cavity, isolating and removing both oviduct structures into 60×15 mm culture plates (CellStar Greiner Bio-One 628160) containing 50 μL droplets of M2+BSA (Millipore MR-015-D supplemented with BSA at 4 mg/mL Sigma 4919, followed by filtration to sterilize with MillexHV SLHV033RB). While viewing through a Stereomicroscope, (Nikon SMZ-U or equivalent), the ampulla of each oviduct is nicked, releasing a mixture of approximately 20 fertilized embryos and unfertilized oocytes surrounded in a cumulus cell network into M2+BSA collection media. All cumulus oocyte complexes are transferred in 50 μL of M2+BSA to a 200 μL droplet of Hyaluronidase/M2 (Millipore MR-051-F) to dissociate cumulus cells from zygotes with an exposure time of approximately 1 minute. All embryos from this point on are manipulated by mouth-pipetting with the use of a 15-inch aspirator tube (Sigma A5177), and a hand-made glass needle fashioned by glass pulling of capillary tubes (Sigma P0674) over an open flame. Embryos are passed through five washes of M2+BSA to remove cumulus cells. With as little additional volume as is reasonable, embryos are transferred to a 200 μL droplet of Acid Tyrode's Solution (Sigma T1788). As batch to batch variation of Acid Tyrode's solution exists, the exact timing of exposure must be ascertained empirically. This is done by exposure and viewing of about 10 embryos under the stereomicroscope. Embryos were exposed until approximately 15-20% of the Zona Pellucida has been digested, which typically occurs between 30-60 seconds. This thinning of the Zona serves to facilitate transfer of protein and nucleic acids into the embryo. Caution must be used so as to not over treat the embryo, as Acid's Tyrode's exposure can lead to a loss of viability. Following treatment, embryos are transferred to an additional M2+BSA wash droplet and then immediately transferred to a second droplet so as to drastically minimize the embryos exposure to fully concentrated Acid Tyrode's solution. This is followed by two additional M2+BSA washes. Embryos are temporarily stored in a water jacketed, 5% CO₂ incubator at 37° C. and 95% humidity, until time of electroporation.

Electroporation of RNP Complex into Embryos

Per electroporation condition, 30 embryos are passed through 10 μL of pre-warmed Opti-MEM Reduced Serum Media (Thermo Fisher Scientific 31985062) a total of three times to dilute M2+BSA volume. In 10 μL of Opti-MEM, all 30 embryos are transferred to 10 uL of Cas9 RiboNucleoProtein (RNP) Mixture. The RNP mixture consisted of 40 μM stock solution of Cas9 Protein in a 1:1.2 molar ratio with sgRNA in 20 mM HEPES PH7.5 (SIGMA h3375), 150 mM KCL (SIGMA p9333), 1 mM MgCl₂ (SIGMA m8266), 10% glycerol (FISHER BP229) and 1 mM TCEP (tris(2-carboxyethyl)phosphine SIGMA c4706) a reducing agent. When required, 200 pmol of HDR template is included. Donor HDR oligos used were: Tyr ssDNA donor v1 5′ GTG CAC CAT CTG GAC CTC AGT TCC CCT TCA AAG GGG TGG ATG ACC GTG AAT TCC TGG CCC TCT GTG TTT TAT AAT AGG ACC TGC CAG TGC TC (SEQ ID NO:1152); Mecp2-L2-loxP 5′CCA GCA ACC TAA AGC TGT TAA GAA ATC TTT GGG CCC CAG CTT GAC CCA AGG ATA CAG TAT GCT AGC ATA ACT TCG TAT AAT GTA TGC TAT ACG AAG TTA TCC TAG GGA AGT TAC CAA AAT CAG AGA TAG TAT GCA GCA GCC AGG GGT CTC ATG TGT GGC A (SEQ ID NO:1153). Mecp2-R1-loxP 5′CCA CTC CTC TGT ACT CCC TGG CTT TTC CAC AAT CCT TAA ACT GAA GGA GTG AGG TCT AGT ATA ACT TCG TAT AGC ATA CAT TAT ACG AAG TTA TGA ATT CAC TTG GGG GTC ATT GGG CTA GAC TGA ATA TCT TTG GTT GGT ACC CAG ACC TAA TCC ACC A (SEQ ID NO: 1154). The RNP Mixture is prepared by incubating at 37° C. for 10 min immediately prior to combining with Embryo/Opti-MEM sample. Entire 20 μL mixture is pipetted into a 1 mm Electroporation cuvette (BIORAD 1652089) and loaded into electroporator (BIORAD Gene Pulser Xcell). Electrical pulse is delivered to the reaction mixture through the square wave delivery protocol. The conditions of the pulse delivery is two pulses at 30V at a pulse length of 3 msec with an interval of 1 msec. Immediately following electroporation, embryos are recovered from the cuvette by flushing with 100 uL of prewarmed KCl-enriched simplex optimization medium with amino acid supplement (KSOM+AA, Zenith Biotech ZEKS-050). An additional 100 uL flush can be used to recover any remaining embryos. Embryos are then washed three times through KSOM+BSA that has been equilibrated prior to the start of the experiment. To equilibrate, 20 uL droplets are prepared in 35×10 mm (CellStar Greiner Bio-One 627160) culture plates and allowed to incubate overnight. Embryos and KSOM+BSA are cultured in a water jacketed, 5% CO₂ incubator at 37° C. and 95% humidity.

### Vasectomy of Male Mice

Male Mice (C57BL/6J JAX 000664), between 3-8 mo of ages, mice are anesthetized with Ketamine 65 mg/kg+Xylazine 13 mg/kg+accepromazine 2 mg/kg mix in sterile 0.9% NaCl solution and place on their backs to expose the abdomen when deeply narcotized. The abdomen is cleaned with 70% ethanol, and a 1.0 cm transverse incision is made in the ventro-distal abdomen to expose the fat pads that overlay the testis and vas deferens. The fatpads are grasped using sterile forceps to further expose both vas deferentia, which are then cauterized. Testis, fat pads and vas deferentia are replaced back into the abdominal cavity. Following this, the abdominal wall is sutured with 3-0 or 4-0 PDS-II taper. The skin incision is then closed using surgical staples. Post-surgical care includes close monitoring and a heating pad to avoid hypothermia until the male awakens from anesthesia. To test fertility, males are mated to supoerovulated or naturally ovulated females. A minimum of two plugged non-pregnant females are required to indicate a successful vasectomy.

Implantation of Embryos into PseudoPregnant Female Mice

Females are placed with vasectomized males and copulation plugs are checked in the next morning o. Plugged females are anesthetized with Ketamine 65 mg/kg+Xylazine 13 mg/kg+accepromazine 2 mg/kg mix in sterile 0.9% NaCl solution, and placed on their stomachs in order to expose the lumbar area for surgery. Fur over the left or right lumbar area is sprayed with 70% ethanol, where a 1 cm or smaller incision is made with sterile scissors. The fat pad overlaying the ovary is grasped with a sterile pair of forceps and pulled until the fatpad, ovary, oviduct and distal end of the uterine horn is exteriorized. 2-cell embryos can be transferred into the oviduct via the infundibulum. The tip of the glass transfer pipette is inserted into the infundibulum, and gentle pressure is applied to place embryos into the oviduct. Following transfer, the incision is sutured and female mouse is monitored.

### Results

### CRISPR-EZ Efficiently Generates Indel Mutations by the NHEJ Repair Pathway

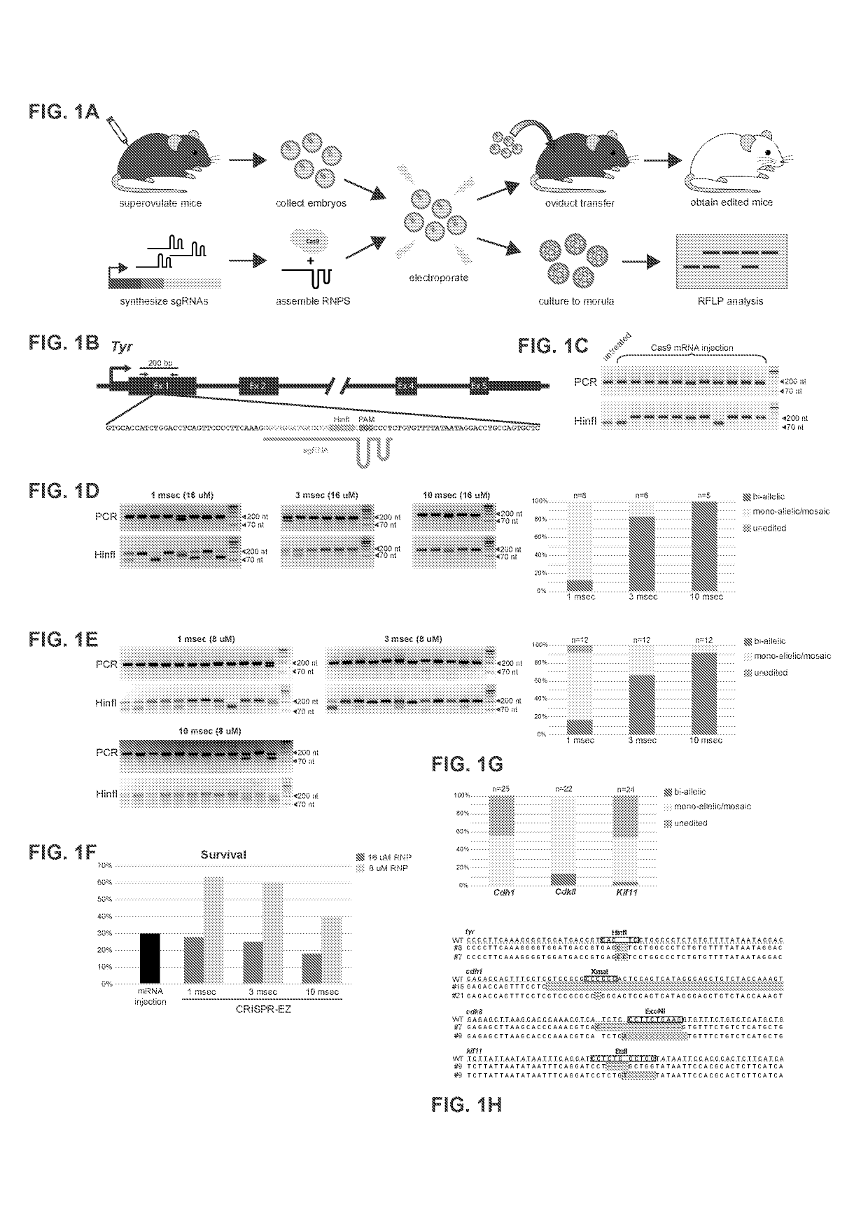

To overcome the costly and laborious nature of the microinjection-based technology, CRISPR-EZ, a highly accessible, electroporation-based method, was developed to deliver Cas9/sgRNA RNP complex in mouse zygotes for in vivo genome editing. Prior to electroporation, C57B6/J mouse zygotes were collected from the oviducts of superovulated female mice, briefly treated with hyaluronidase to remove cumulus cells, and washed for 30 seconds with acid Tyrode's solution to weaken the zona pellucida. ˜30-40 pre-treated mouse zygotes were then combined with preassembled Cas9/sgRNA RNP complexes for electroporation (e.g., 30V, 1 ms pulse duration, 2 pulses, 1 ms pulse interval). Finally, electroporated embryos were either cultured to the 2-cell stage before transferred to the oviducts of pseudopregnant recipient females or cultured to the morula stage for genotyping analysis (FIG. 1A).

To optimize electroporation conditions for efficient Cas9/sgRNA RNP delivery into mouse zygotes, a sgRNA was selected, which sgRNA induces NHEJ-mediated mutations into exon 1 of the tyr gene (FIG. 1B)⁴⁰, which is predicted to ablate a HinfI restriction site 1 nt upstream of the Protospacer Adjacent Motif (PAM) (FIG. 1C). The genome editing efficiency and embryo survival rates were determined in CRISPR-EZ experiments at various RNP concentrations (16 μM or 8 μM) and electroporation pulse lengths (1 millisecond (msec), 3 msec, or 10 msec) (FIGS. 1D and 1E). Electroporated embryos were cultured to the morula stage and subjected to a restriction fragment length polymorphism (RFLP) assay for genotyping (FIG. 1A). While CRISPR-EZ at 1 msec pulse length yielded mostly partially edited embryos, 3 msec and 10 msec conditions resulted in mostly bi-allelic editing that were sequence confirmed (83-100%, FIGS. 1D and 1E; Table 1). Notably, 3 msec and 10 msec conditions left no unedited embryos, indicating a 100% efficiency in Cas9/sgRNA RNP delivery, yet the 10 msec pulse condition, but not the 3 msec pulse condition, reduced embryo viability (Table 1). Additionally, a high RNP concentration also negatively impacts embryo survival. At the 3 msec pulse condition, the 8 μM and 16 μM RNP concentrations both resulted in mostly bi-allelic editing (67% and 83% respectively), but the 8 μM condition enabled 2.4-fold greater embryo survival (60% and 25%, respectively). Thus, using 8 μM Cas9/sgRNA RNP for electroporation at a single pulse length of 3 msec achieved the best balance between CRISPR editing efficiency and embryo survival (67% bi-allelic editing and 60% embryo survival). Compared to microinjection-based technology, this optimized CRISPR-EZ condition yields a comparable editing efficiency, yet significantly improve the embryo survival rate (60% for CRISPR-EZ versus 30% for microinjection) (FIG. 1F).

To evaluate the robustness of the CRISPR-EZ technology, three additional genes, cdh1, cdk8, and kif11, were edited in mouse zygotes. In each case, sgRNAs were designed to target a restriction site 3-4 nucleotides (nt) upstream of the PAM, thus allowing us to assess NHEJ editing efficiency by RFLP analyses. While CRISPR-EZ editing efficiency varies with the different sgRNA design, at least 50% of mouse embryos exhibited desired editing for each gene (FIG. 1G, Table 2), which were subsequently confirmed by sequencing (FIG. 1H). Thus, CRISPR-EZ efficiently delivers Cas9/sgRNA RNP complexes to introduce indel mutations through the NHEJ repair pathway.

FIG. 1A-1H. CRISPR-EZ Generates NHEJ-Mediated Indel Mutations.

A. Overview of CRISPR-EZ and RFLP analysis Workflow. Fertilized embryos are combined with pre-assembled Cas9/sgRNA RiboNucleoProtein (RNP) complexes prior to electroporation. Following this, embryos were either cultured to the morula stage of preimplantation development to assess for editing efficiency via restriction fragment length polymorphism assay, or embryos are transferred to pseudopregnant females to generate edited animals. B. Diagram of tyr gene structure, sgRNA design and Genotyping Strategy. The sgRNA (orange) hybridizes within the open reading frame in exon 1. A HinfI restriction site is located 1 nt upstream of the protospacer adjacent motif (PAM), where Cas9 is predicted to cleave. Upon successful Non Homologous End Joining (NHEJ) repair outcomes, this restriction site is predicted to be disrupted and no longer a substrate for HinfI. Arrows indicate position of primers used for polymerase chain reaction (PCR). C. Representative outcome of genotyping strategy applied to a Cas9 mRNA microinjection of embryo based editing approach. Embryos were lysed at the morula stage, subjected to nested PCR, and digested with HinfI for 2 hours. Complete digestion by HinfI generates two ˜100 nt digestion products that migrate together as a single lower band. Absence of this lower band was used to determine the degree of editing. Presence of both digested and undigested product suggest mono-allelic or mosaic editing events. Top: PCR amplicons from 1 control (unedited) and 11 recovered Morula staged embryos following microinjection of Cas9 mRNA+sgTyr sgRNA at the pronucleus stage. Bottom: RFLP analysis using HinfI restriction enzyme of identical nested PCR amplicons as top part of image. D. Determining optimal pulse length. Three different electroporation pulse conditions were compared with constant RNP concentration (16 μM). Analysis was performed as described in C. Quantification of results are displayed on right-most panel. E. Determining optimal pulse length at lower RNP concentration. Three different electroporation pulse conditions were compared with constant RNP concentration (8 μM). Analysis was performed as described in previous panel. Quantification of results is displayed on right-most panel. F. Comparison of embryo viability following sgRNA/Cas9 mRNA microinjection and Electroporation of RNP complex at various pulse length and RNP concentration conditions. Percent survival was assessed by first determining the number of embryos that were able to reach the 2-Cell stage (evidence for fertilization), and subsequently the number of these 2-Cell embryos that developed to the Morula stage without arresting prior to collection. G. RFLP analysis of editing efficiency of sgRNAs targeting Cdh1, Cdk8 and Kif11. The efficiency of three additional sgRNAs was tested using the optimized conditions determined above to yield highest editing and viability of electroporated embryos. Restriction enzymes used to determine editing efficiencies were XmaI for Cdh1, EcoNI for Cdk8 and BsII for Kif11. H: Sequence verification of Tyr, Cdh1, Cdk8 and Kif11 sgRNA editing events. Nested PCR products from suspected edited embryos were gel extracted, cloned, and sequenced. The recovered sequences were then aligned to the appropriate unedited sequence to display the NHEJ repair outcome of each sgRNA/Cas9 mediated double strand break. At least two distinct insertion/deletion repair events were recovered for each sgRNA tested.

### CRISPR-EZ Efficiently Generates Precise Mutations by HDR in Live Mice

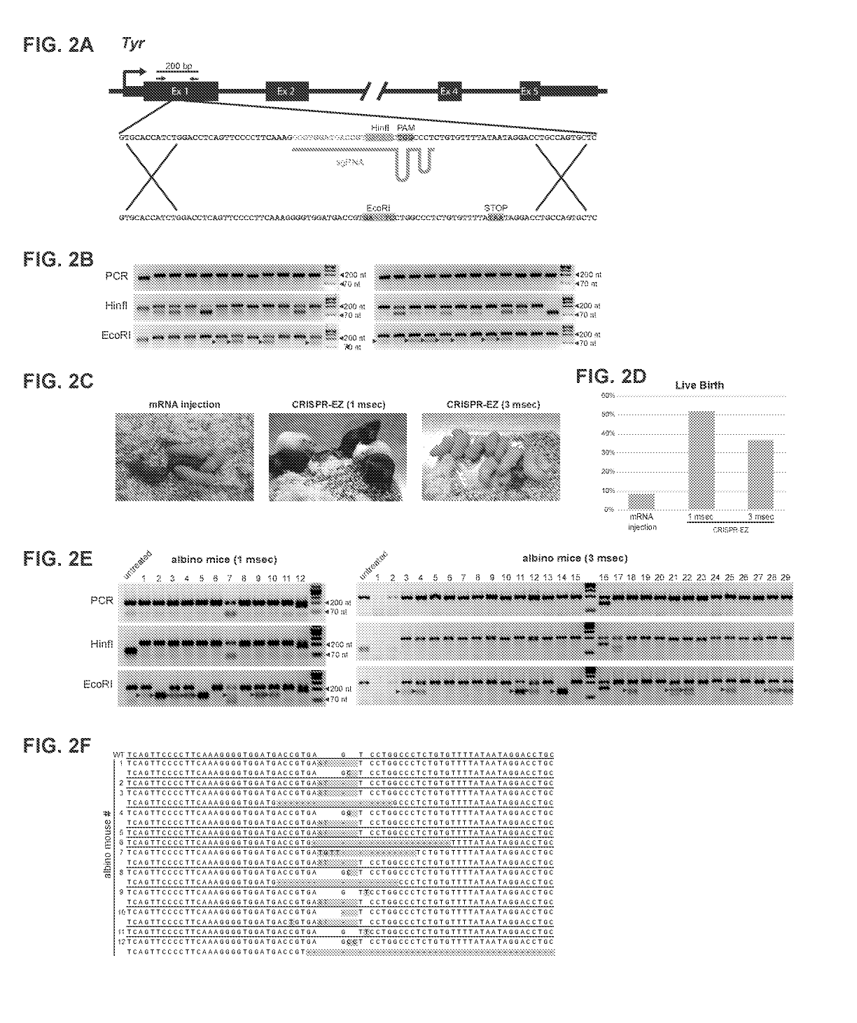

Next, it was determined whether CRISPR-EZ can be employed to introduce specific point mutations through the HDR pathway. A 92 nt ssDNA donor oligonucleotide (“oligo”) was designed, which oligo enables the substitution of the endogenous HinfI site for an EcoRI site in tyr exon1, causing an early termination of the open reading frame (ORF) and generating a null tyr allele (FIG. 2A). Purified Cas9 protein, in vitro transcribed sgRNA, and the ssDNA donor were combined to assemble RNPs, and obtained ˜46% efficiency for HDR in cultured morula embryos in CRISRP-EZ experiments. (FIG. 2B, also see FIG. 2G).

Tyrosinase is the rate-limiting enzyme in pigment biosynthesis, thus the extent of the albino coat color in mice is a direct readout of the efficiency of bi-allelic tyr inactivation in vivo. Any mosaicism in editing will be accurately reflected in the mosaicism of the coat color. CRISPR-EZ was performed to generate live animals that harbor the HDR-mediated tyr gene modification as described above. CRISPR-EZ was performed using 1 msec and 3 msec pulse lengths to electroporate Cas9/sgRNA RNP with donor DNA into 140 and 120 zygotes, respectively. Electroporated zygotes were then incubated in KSOM for 24 hours to reach 2-cell stage embryos, and viable 2-cell embryos were transferred to the oviducts of pseudopregnant foster mothers. The 3 msec CRISPR-EZ pulse length condition is highly efficient in genome editing, generating 88% albino mice with bi-allelic tyr editing (29/33), 9% (3/33) mosaic mice with ˜50% albino coat and 3% mouse with a partial tyr editing (FIG. 2C, Table 3). All tested edited mice are germline competent. Using RFLP analyses on isolated tail DNA, it was validated that 42% of animals harbored the HDR-mediated precise modifications (FIG. 2F). Remarkably, generated homozygous HDR-edited mice were generated that were germline competent. In comparison, the 1 msec CRISPR-EZ pulse condition, while slightly increasing the live birth rate (Table 3), only yield 41% (18/44) albino mice and 27% HDR-mediated editing (FIG. 2C, 2F, Table 3). Nevertheless, both CRISPR-EZ conditions offer a significant improvement on embryo survival compared to the microinjection-based technology to deliver Cas9 mRNA and sgRNA, and the 3 msec CRISPR-EZ pulse length condition also improves on editing efficiency as measured by the percentage of albino animals generated. Thus, CRISPR-EZ generates HDR-edited, germline competent mice with unprecedented speed and efficiency.

In addition to small sequence replacement, the CRISPR-EZ technology can also be employed to generate precise deletion or introduce a small insertion. CRISPR-EZ technology has been successfully employed to generate a ˜700 bp deletion in MeCP2 gene with nearly 70% efficiency. In addition, genetically modified mouse embryos have been generated with an insertion of a V5 tag in the oct4 gene. Taken together, CRISPR-EZ yield a greater editing efficiency, a greater embryo survival and live birth rate in in vivo genome editing, and can replace microinjection-based technology for CRISPR editing in a variety of mammalian species.

FIGS. 2A-2F. CRISPR-EZ generates HDR-mediated precise point mutations in live animals. A. Diagram of HDR targeting strategy. A 92 nt single-stranded DNA donor that substitutes the HinfI site for an EcoRI site was co-electroporated along with RNPs. Successful HDR results in a frameshift mutation leading to early termination of the polypeptide 18 nt downstream of the EcoRI site. B. Treated embryos were lysed at the morula stage, subjected to nested PCR, and digested with HinfI or EcoRI for 2 hours. Black arrows mark EcoRI digestion products, indicating HDR-mediated sequence substitution. C. Images of mouse litters obtains from CRISPR-EZ 1 msec pulse condition (left) and 3 msec pulse condition (right). D. Restriction analysis was performed using tail samples from albino mice. White arrows mark EcoRI digestion products, indicating HDR-mediated sequence substitution. E. PCR products from 1 msec pulse condition albino mice were cloned into sequencing vectors, and 8 clones from each animal were picked for sequencing. F. All sequence variants are shown for each animal, with edited sequences highlighted in pink. Red “AT” indicate sequences introduced by HDR.

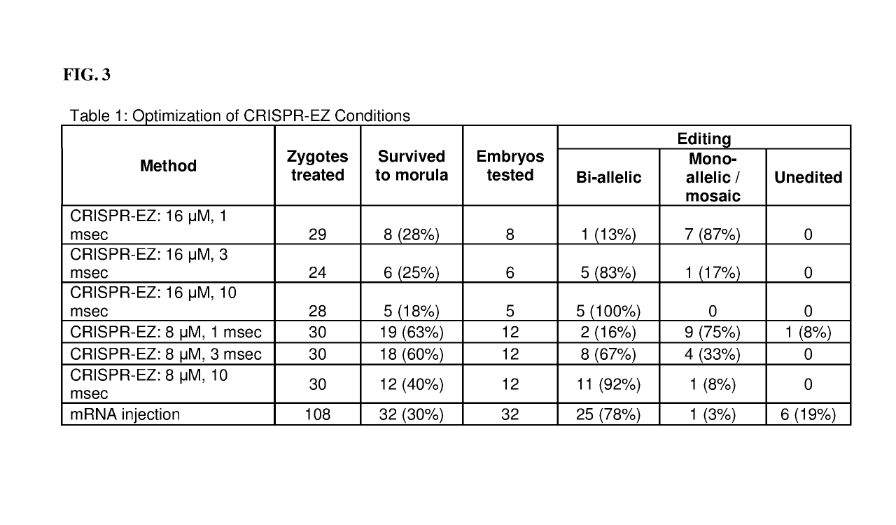

Table 1 (provided in FIG. 3). Optimization of CRISRP-EZ conditions. Cas9 protein and sgRNAs were assembled at 1:1.5 molar ratio and embryos were electroporated at a final concentration of 16 μM or 8 μM. Embryos were electroporated in pools of 30 embryos using 1 msec, 3 msec, or 10 msec pulse lengths, with other parameters held constant: 2 pulses, 30 volts, 1 msec interval. Electroporated embryos were transferred to KSOM and incubated for 3 days, followed by lysis, nested PCR, and RFLP analysis. For microinjection, Cas9 mRNA and sgRNA were co-injected at 100 ng/μL and 50 ng/μL respectively, with approximately 4-5 pL injected per embryo.

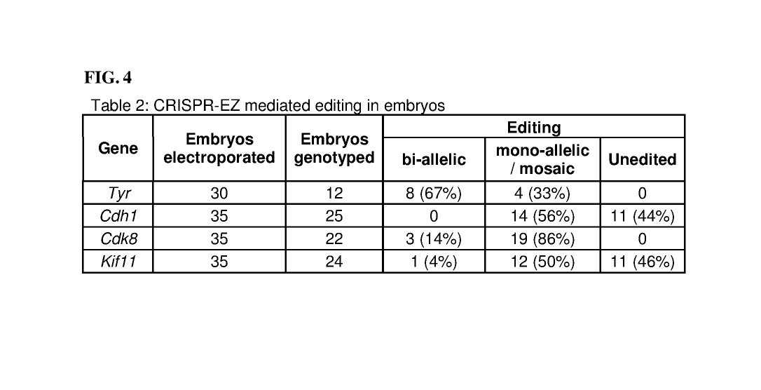

Table 2 (provided in FIG. 4). CRISPR-EZ mediated editing in embryos. Cas9 protein and sgRNAs were assembled at 1:1.5 molar ratio and embryos were electroporated at a final concentration of 8 μM. Embryos were electroporated in pools of 30-35 embryos using the following conditions: 2 pulses, 3 msec pulse length, 30 volts, 1 msec interval. Electroporated embryos were transferred to KSOM and incubated for 3 days, followed by lysis, nested PCR, and RFLP analysis.

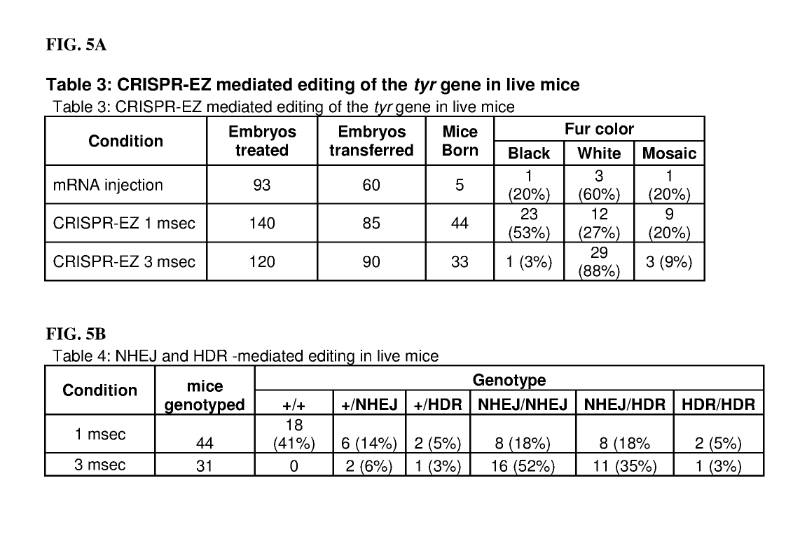

Table 3 (provided in FIG. 5A). CRISPR-EZ mediated editing of the tyr gene in live mice. Cas9 protein and sgRNAs were assembled at 1:1.5 molar ratio and embryos were electroporated at a final concentration of 8 μM. Embryos were electroporated in pools of 35 embryos using 1 msec or 3 msec pulse lengths, with other parameters held constant: 2 pulses, 30 volts, 1 msec interval. Electroporated embryos were cultured in KSOM for 24 hours before transferring the 2-cell stage embryos to the oviducts of pseudopregnant foster mothers. For microinjection, Cas9 mRNA and sgRNA were co-injected at 100 ng/μL and 50 ng/uL respectively, with approximately 4-5 pL injected per embryo.

Table 4 (provided in FIG. 5B). NHEJ and HDR-mediated editing in live mice. Tail DNA was recovered from all CRISPR-EZ edited mice generated using either a 1 msec or 3 msec pulse length protocol. DNA was amplified by nested PCR and subjected to RFLP analysis using HinfI and EcoRI to determine the genotypes of mice.

### REFERENCES

1. Mansour, S. L., Thomas, K. R. & Capecchi, M. R. Disruption of the proto-oncogene int-2 in mouse embryo-derived stem cells: a general strategy for targeting mutations to non-selectable genes. Nature 336, 348-52 (1988).
2. Evans, M. J. & Kaufman, M. H. Establishment in culture of pluripotential cells from mouse embryos. Nature 292, 154-156 (1981).
3. Capecchi, M. R. Gene targeting in mice: functional analysis of the mammalian genome for the twenty-first century. Nat. Rev. Genet. 6, 507-12 (2005).
4. Geurts, A. M. et al. Knockout rats via embryo microinjection of zinc-finger nucleases. Science 325, 433 (2009).
5. Carbery, I. D., Ji, D., Harrington, A., Brown, V., Weinstein, E. J., Liaw, L. & Cui, X. Targeted genome modification in mice using zinc-finger nucleases. Genetics 186, 451-9 (2010).
6. Tesson, L., Usal, C., Ménoret, S., Leung, E., Niles, B. J., Remy, S., Santiago, Y., Vincent, A. I., Meng, X., Zhang, L., Gregory, P. D., Anegon, I. & Cost, G. J. Knockout rats generated by embryo microinjection of TALENs. Nat. Biotechnol. 29, 695-6 (2011).
7. Sung, Y. H., Baek, I.-J., Kim, D. H., Jeon, J., Lee, J., Lee, K., Jeong, D., Kim, J.-S. & Lee, H.-W. Knockout mice created by TALEN-mediated gene targeting. Nat. Biotechnol. 31, 23-4 (2013).
8. Meyer, M., de Angelis, M. H., Wurst, W. & Kühn, R. Gene targeting by homologous recombination in mouse zygotes mediated by zinc-finger nucleases. Proc. Natl. Acad. Sci. U.S.A. 107, 15022-6 (2010).
9. Cui, X., Ji, D., Fisher, D. A., Wu, Y., Briner, D. M. & Weinstein, E. J. Targeted integration in rat and mouse embryos with zinc-finger nucleases. Nat. Biotechnol. 29, 64-7 (2011).
10. Carroll, D. Genome engineering with zinc-finger nucleases. Genetics 188, 773-82 (2011).
11. Barrangou, R., Fremaux, C., Deveau, H., Richards, M., Boyaval, P., Moineau, S., Romero, D. A. & Horvath, P. CRISPR provides acquired resistance against viruses in prokaryotes. Science 315, 1709-12 (2007).
12. Brouns, S. J. J., Jore, M. M., Lundgren, M., Westra, E. R., Slijkhuis, R. J. H., Snijders, A. P. L., Dickman, M. J., Makarova, K. S., Koonin, E. V & van der Oost, J. Small CRISPR RNAs guide antiviral defense in prokaryotes. Science 321, 960-4 (2008).
13. Xiao, A., Wang, Z., Hu, Y., Wu, Y., Luo, Z., Yang, Z., Zu, Y., Li, W., Huang, P., Tong, X., Zhu, Z., Lin, S. & Zhang, B. Chromosomal deletions and inversions mediated by TALENs and CRISPR/Cas in zebrafish. Nucleic Acids Res. 41, e141 (2013).
14. Niu, Y. et al. Generation of gene-modified cynomolgus monkey via Cas9/RNA-mediated gene targeting in one-cell embryos. Cell 156, 836-43 (2014).
15. Guo, X., Zhang, T., Hu, Z., Zhang, Y., Shi, Z., Wang, Q., Cui, Y., Wang, F., Zhao, H. & Chen, Y. Efficient RNA/Cas9-mediated genome editing in Xenopus tropicalis. Development 141, 707-14 (2014).
16. Waaijers, S., Portegijs, V., Kerver, J., Lemmens, B. B. L. G., Tijsterman, M., van den Heuvel, S. & Boxem, M. CRISPR/Cas9-targeted mutagenesis in Caenorhabditis elegans. Genetics 195, 1187-91 (2013).
17. Gokcezade, J., Sienski, G. & Duchek, P. Efficient CRISPR/Cas9 plasmids for rapid and versatile genome editing in Drosophila. G3 (Bethesda). 4, 2279-82 (2014).
18. Jinek, M., Chylinski, K., Fonfara, I., Hauer, M., Doudna, J. A. & Charpentier, E. A programmable dual-RNA-guided DNA endonuclease in adaptive bacterial immunity. Science 337, 816-21 (2012).
19. Cong, L., Ran, F. A., Cox, D., Lin, S., Barretto, R., Habib, N., Hsu, P. D., Wu, X., Jiang, W., Marraffini, L. A. & Zhang, F. Multiplex genome engineering using CRISPR/Cas systems. Science 339, 819-23 (2013).
20. Wang, H., Yang, H., Shivalila, C. S., Dawlaty, M. M., Cheng, A. W., Zhang, F. & Jaenisch, R. One-step generation of mice carrying mutations in multiple genes by CRISPR/cas-mediated genome engineering. Cell 153, 910-918 (2013).
21. Maddalo, D., Manchado, E., Concepcion, C. P., Bonetti, C., Vidigal, J. A., Han, Y.-C., Ogrodowski, P., Crippa, A., Rekhtman, N., de Stanchina, E., Lowe, S. W. & Ventura, A. In vivo engineering of oncogenic chromosomal rearrangements with the CRISPR/Cas9 system. Nature 516, 423-427 (2014).
22. Canver, M. C., Bauer, D. E., Dass, A., Yien, Y. Y., Chung, J., Masuda, T., Maeda, T., Paw, B. H. & Orkin, S. H. Characterization of genomic deletion efficiency mediated by clustered regularly interspaced palindromic repeats (CRISPR)/Cas9 nuclease system in mammalian cells. J. Biol. Chem. 289, 21312-24 (2014).
23. Yang, H., Wang, H., Shivalila, C. S., Cheng, A. W., Shi, L. & Jaenisch, R. One-step generation of mice carrying reporter and conditional alleles by CRISPR/Cas-mediated genome engineering. Cell 154, 1370-9 (2013).
24. Irion, U., Krauss, J. & Nüsslein-Volhard, C. Precise and efficient genome editing in zebrafish using the CRISPR/Cas9 system. Development 141, 4827-30 (2014).
25. Gratz, S. J., Cummings, A. M., Nguyen, J. N., Hamm, D. C., Donohue, L. K., Harrison, M. M., Wildonger, J. & O'Connor-Giles, K. M. Genome engineering of Drosophila with the CRISPR RNA-guided Cas9 nuclease. Genetics 194, 1029-35 (2013).
26. Bassett, A. R., Tibbit, C., Ponting, C. P. & Liu, J.-L. Highly Efficient Targeted Mutagenesis of Drosophila with the CRISPR/Cas9 System. Cell Rep. 6, 1178-1179 (2014).
27. Wang, H., Yang, H., Shivalila, C. S., Dawlaty, M. M., Cheng, A. W., Zhang, F. & Jaenisch, R. One-step generation of mice carrying mutations in multiple genes by CRISPR/Cas-mediated genome engineering. Cell 153, 910-8 (2013).
28. Qin, W., Dion, S. L., Kutny, P. M., Zhang, Y., Cheng, A., Jillette, N. L., Malhotra, A., Geurts, A. M., Chen, Y.-G. & Wang, H. Efficient CRISPR/Cas9-Mediated Genome Editing in Mice by Zygote Electroporation of Nuclease. Genetics (2015). doi:10.1534/genetics.115.176594
29. Takahashi, G., Gurumurthy, C. B., Wada, K., Miura, H., Sato, M. & Ohtsuka, M. GONAD: Genome-editing via Oviductal Nucleic Acids Delivery system: a novel microinjection independent genome engineering method in mice. Sci. Rep. 5, 11406 (2015).
30. Hashimoto, M. & Takemoto, T. Electroporation enables the efficient mRNA delivery into the mouse zygotes and facilitates CRISPR/Cas9-based genome editing. Sci. Rep. 5, 11315 (2015).
31. Yen, S.-T., Zhang, M., Deng, J. M., Usman, S. J., Smith, C. N., Parker-Thornburg, J., Swinton, P. G., Martin, J. F. & Behringer, R. R. Somatic mosaicism and allele complexity induced by CRISPR/Cas9 RNA injections in mouse zygotes. Dev. Biol. 393, 3-9 (2014).
32. Gasiunas, G., Barrangou, R., Horvath, P. & Siksnys, V. Cas9-crRNA ribonucleoprotein complex mediates specific DNA cleavage for adaptive immunity in bacteria. Proc. Natl. Acad. Sci. U.S.A 109, E2579-86 (2012).
33. Nishimasu, H., Ran, F. A., Hsu, P. D., Konermann, S., Shehata, S. I., Dohmae, N., Ishitani, R., Zhang, F. & Nureki, O. Crystal structure of Cas9 in complex with guide RNA and target DNA. Cell 156, 935-49 (2014).
34. Jinek, M., Jiang, F., Taylor, D. W., Sternberg, S. H., Kaya, E., Ma, E., Anders, C., Hauer, M., Zhou, K., Lin, S., Kaplan, M., Iavarone, A. T., Charpentier, E., Nogales, E. & Doudna, J. A. Structures of Cas9 endonucleases reveal RNA-mediated conformational activation. Science 343, 1247997 (2014).
35. Cho, S. W., Lee, J., Carroll, D., Kim, J.-S. & Lee, J. Heritable gene knockout in Caenorhabditis elegans by direct injection of Cas9-sgRNA ribonucleoproteins. Genetics 195, 1177-80 (2013).
36. Lee, J.-S., Kwak, S.-J., Kim, J., Kim, A.-K., Noh, H. M., Kim, J.-S. & Yu, K. RNA-guided genome editing in Drosophila with the purified Cas9 protein. G3 (Bethesda). 4, 1291-5 (2014).
37. Kim, S., Kim, D., Cho, S. W., Kim, J. & Kim, J.-S. Highly efficient RNA-guided genome editing in human cells via delivery of purified Cas9 ribonucleoproteins. Genome Res. 24, 1012-9 (2014).
38. Lin, S., Staahl, B., Alla, R. K. & Doudna, J. A. Enhanced homology-directed human genome engineering by controlled timing of CRISPR/Cas9 delivery. Elife 3, e04766 (2014).
39. Schumann, K., Lin, S., Boyer, E., Simeonov, D. R., Subramaniam, M., Gate, R. E., Haliburton, G. E., Ye, C. J., Bluestone, J. A., Doudna, J. A. & Marson, A. Generation of knock-in primary human T cells using Cas9 ribonucleoproteins. Proc. Natl. Acad. Sci. 112, 201512503 (2015).
40. Mizuno, S., Dinh, T. T. H., Kato, K., Mizuno-Iijima, S., Tanimoto, Y., Daitoku, Y., Hoshino, Y., Ikawa, M., Takahashi, S., Sugiyama, F. & Yagami, K. Simple generation of albino C57BL/6J mice with G291T mutation in the tyrosinase gene by the CRISPR/Cas9 system. Mamm. Genome 25, 327-34 (2014).

### Example 2: Deletion of a Retrotransposon Found Upstream of Cdk2ap1

To investigate a potential regulatory and structural effect elicited on the protein coding gene Cdk2ap1 by the nearby and upstream retrotransposon (RT) element MT2C_Mm, a CRISPR/Cas9-based genome editing strategy was developed to remove the RT as well as any effect it might have on Cdk2ap1.

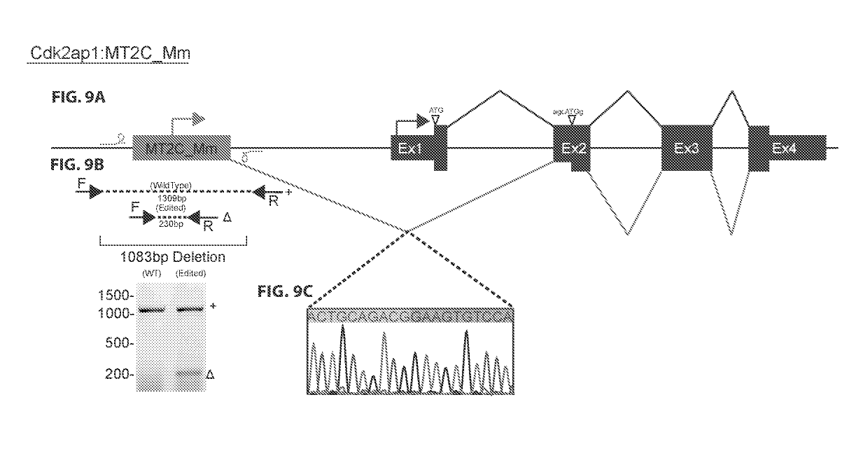

The hypothesized relationship between Cdk2ap1 and MT2C_Mm that is to be disrupted is one in which the RT sequence has been co-opted by the genome as an alternative promoter and 5′ UTR for Cdk2ap1. In addition to harboring appropriately utilized splicing signals, the novel chimeric splice isoform enables the use of a downstream start codon, effectively truncating the protein product by 27 amino acids, while leaving the remaining downstream 87 amino acids intact and in-frame. Using a pair of small guide RNAs (sgRNAs) flanking the RT, a 1083 bp deletion is generated. This event will be tracked using primers designed to distinguish edited and unedited cells and tissues (FIG. 9B). As additional evidence of the presence of this particular chimeric transcript, primers designed to target this specific splicing event were generated and used on a cDNA sample template predicted to possess this isoform. The resulting amplicon was isolated and subcloned for sequencing analysis. The predicted splicing event was recovered in precisely the manner predicted.

### Example 3: CRISPR-EZ Efficiency In Vivo

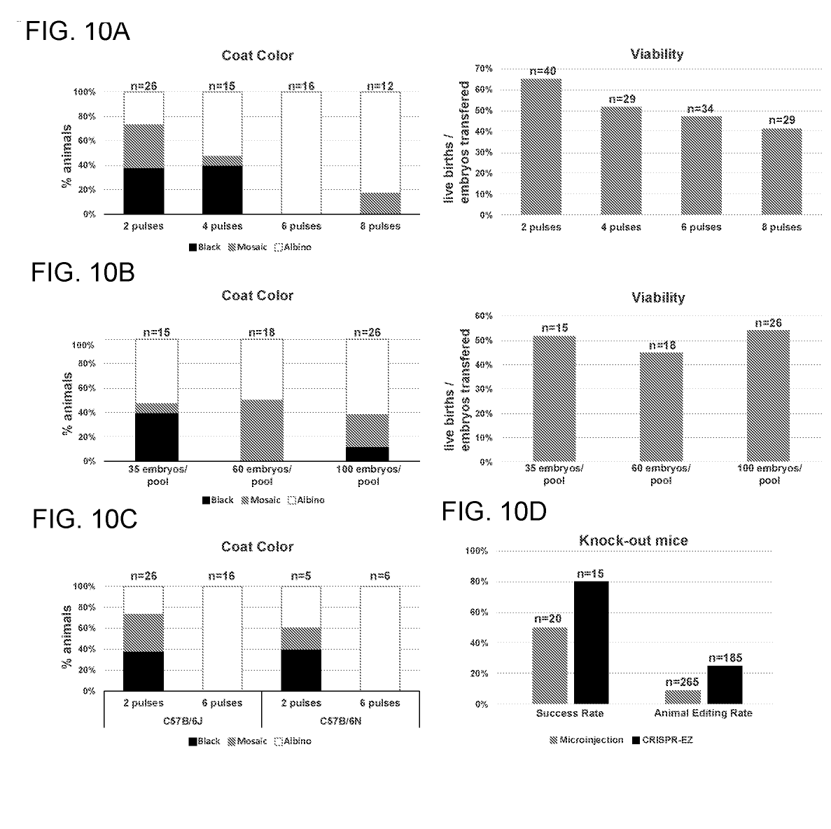

A variety of experimental conditions to test CRISPR-EZ efficiency in mice have been performed. In addition to optimizing Cas9 concentration and pulse length in vitro, optimization in vivo the number of electroporation pulses required to achieve the best balance between editing efficiency and animal viability was tested. Using a Tyr targeting strategy, CRISPR-EZ was performed using 2, 4, 6, or 8 pulses (30 volts, 3 msec) followed by transfer of the electroporated embryos into pseudopregnant recipient females. Coat color of the resulting animals was quantified to determine editing efficiency: an albino coat indicates complete biallelic editing, a mosaic coat containing patches of white and black indicates biallelic Tyr disruption in only some cells, and a black coat indicates heterozygous or unedited animals. For the 6-pulse condition, 16/16 live animals were completely albino, suggesting 100% biallelic disruption of Tyr (FIG. 10A). Furthermore, this condition did not appreciably compromise animal viability—out of 34 embryos transferred, 16 pups were born (47%) (FIG. 10A). Thus, 6 pulses provide a balance of editing efficiency and animal viability for C57B6/J strain mice.

Multiple embryos can be simultaneously electroporated in one cuvette at the push of a button. In contrast, a microinjection experiment can take hours even in the hands of a skilled technician, since zygotes must be individually injected one at a time. To test the throughput of CRISPR-EZ, the Tyr gene was targeted on pools of 35, 60, or 100 embryos in a single cuvette, using the following electroporation conditions: 30 volts, 3 msec, 4 pulses. Up to 100 embryos could be simultaneously treated without a reduction in editing efficiency or viability (FIG. 10B).

The next steps were to reproduce the results from C57B/6J mice in a different yet related C57B/6N strain. For both 2 pulse and 6 pulses conditions, editing of the Tyr gene in C57B/6N closely matched efficiencies obtained from C57B/6J, demonstrating that CRISPR-EZ can be applied to other mouse strains with minimal optimization (FIG. 10C). The CRISPR-EZ was able to adapt to different mouse strains.

Next, the robustness of CRISPR-EZ vs. microinjection in generating knock-out mice in a high throughput manner was compared. In this experiment, Cas9 RNPs consisting of 4 sgRNAs (2 upstream and 2 downstream) flanking a key exon were introduced into zygotes by CRISPR-EZ or pronuclear microinjection, followed by embryo transfer into pseudopregnant recipient females to generate live animals. Gene editing resulted in deletion of the intervening sequences, which was genotyped by PCR and sequencing of tail DNA. CRISPR-EZ outperformed microinjection—while ˜9% of animals were edited by microinjection, ˜25% of animals were edited by CRISPR-EZ (FIG. 10D). Furthermore, ˜50% of genes targeted by microinjection produced at least one correctly edited animal, in contrast with ˜80% by CRISPR-EZ (FIG. 10D). Notably, all these experiments were carried out in C57B/6N strain mice.

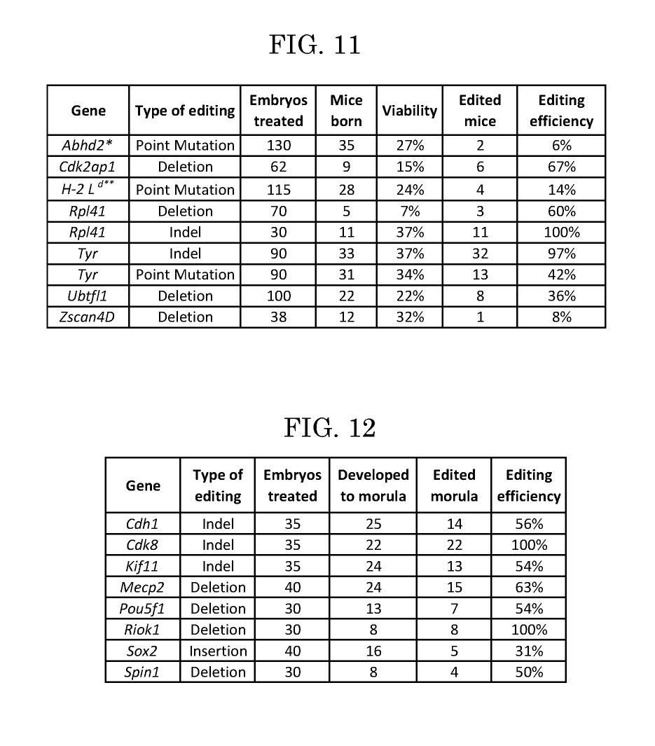

The CRISPR-EZ technique generated multiple genome editing schemes in mice and in embryos, including indels in Cdk8, Cdh1 and Kif11, deletion of putative regulatory elements or gene exons in the Cdk2ap1, Rpl41, Ubtfl1, Zscan4D, MeCP2, Pou5f1, Spin1 genes, insertion of an V5 tag to the Sox2 gene, introduction of point mutations to the Tyr gene (FIG. 11, FIG. 12). CRISPR-EZ was used to produce genetically modified mice to make a point mutation by homology directed repair (HDR) in the major histocompatibility gene H-2 Ld. Additionally, germline competent edited mice using CRISPR-EZ (FIG. 11) were generated. CRISPR-EZ was also used to make a point mutation in the Abhd2 gene using homology directed repair (HDR) (FIG. 11).

FIG. 9A-9C. Deletion of retrotransposon found upstream of Cdk2ap1. A) Schematic of the organization between the Non-Coding Retrotransposon “MT2C_Mm” and the Protein Coding Gene “Cdk2ap1”. Features included, from left to right: Upstream small guide RNA (sgRNA), Annotation of MT2C_Mm including predicted Transcriptional Start Site (TSS), downstream sgRNA, Exon 1 of Cdk2ap1 with TSS and Start Codon (ATG). Exon 2 with alternative Start Codon, Remaining unaltered exons of CDk2ap1. Blue and Red lines represent splice junctions of protein coding exons and RT derived exons, respectively. B) Genotyping strategy for determining the presence or absence of the deleted alleles. C) Sequencing confirmation of splice junction between MT2C_Mm and Exon 2 of CDK2ap1. Ten nucleotides on either side of junction are shown along with chromatogram trace.

FIG. 10A-D. Optimization of CRISPR-EZ efficiency, throughput, and robustness to enhance genome editing efficiency and survival. FIG. 10A. Electroporation pulse number was optimized in CRISPR-EZ experiments using C57B/6J mice. CRISPR-EZ targeting the Tyr gene was performed using 2, 4, 6, or 8 pulses of 30 volts at 3 ms. Electroporated embryos were transferred into pseudopregnant recipient females that gave birth to edited animals. 6 pulses offered maximal editing efficiency (left) as indicated by albino coat color, with minimal reduction in animal viability (right). Coat color of the resulting animals was quantified to determine editing efficiency: an albino coat indicates complete biallelic editing, a mosaic coat containing patches of white and black indicates biallelic Tyr disruption in only some cells, and a black coat indicates heterozygous or unedited animals. FIG. 10B. The number of embryos that can be simultaneously electroporated was investigated using C57B/6J mice. Simultaneous electroporation of 35, 60, or 100 zygotes (30 volts, 3 ms, 4 pulses) was performed in one electroporation cuvette, followed by transfer of a portion of the embryos into recipient females. For up to 100 embryos, there was no observed reduction in editing efficiency (left) or animal viability (right). FIG. 10C. Robustness across different mouse strains for CRISPR-EZ genome editing was tested.

CRISPR-EZ was performed on two different mouse strains (C57B/6J or C57/6N) using 2 or 6 pulses. Similar editing efficiency was achieved for both mouse strains under similar conditions, suggesting that CRISPR-EZ can be adapted to other mouse strains. FIG. 10D. Comparison between CRISPR-EZ and pronuclear microinjection in generating knock-out mice in C57/6N strains. 20 genes and 15 genes were tested by microinjection and CRISPR-EZ, respectively. For each gene, 2 sgRNAs upstream and 2 sgRNAs downstream of a key exon were introduced into zygotes by microinjection or CRISPR-EZ, such that successful editing results in deletion of the targeted exon. Treated embryos were then transferred to recipient females, and editing in the resulting pups was assessed by PCR. “Success rate” is defined as the percent of genes for which at least one edited mouse was obtained. “Animal editing rate” is defined as the percent of animals carrying an edited allele.

FIG. 11 provides a table showing that CRISPR-EZ generates live mice harboring a variety of editing schemes. Zygotes were collected from superovulated females, treated by CRISPR-EZ, and transferred to pseudopregnant recipient females that gave birth to edited mice. Editing was confirmed by sequencing and animals were germline competent.

FIG. 12 provides a table showing that CRISPR-EZ generates a variety of editing schemes in vitro. CRISPR-EZ was performed on zygotes harvested from superovulated females Zygotes were then cultured to morula stage; the morula were subjected to restriction fragment length polymorphism analysis and sequencing to assess editing.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of modifying genomic DNA of a mammalian zygote, the method comprising introducing into the zygote a ribonucleoprotein (RNP) comprising a class 2 CRISPR/Cas endonuclease complexed with a corresponding CRISPR/Cas guide RNA that hybridizes to a target sequence within the genomic DNA of the zygote, wherein said introducing is by electroporation of an electroporation composition comprising the RNP and the zygote, and wherein said introducing results in modification of the genomic DNA.

2. The method of claim 1, wherein the class 2 CRISPR/Cas endonuclease is a type II CRISPR/Cas endonuclease.

3. The method of claim 1 or claim 2, wherein the class 2 CRISPR/Cas endonuclease is a Cas9 polypeptide and the corresponding CRISPR/Cas guide RNA is a Cas9 guide RNA.

4. The method of claim 3, wherein the Cas9 guide RNA is a single guide RNA (sgRNA).

5. The method of claim 1, wherein the RNP comprises two or more CRISPR/Cas guide RNAs.

6. The method of claim 1, wherein the class 2 CRISPR/Cas endonuclease is a type V or type VI CRISPR/Cas endonuclease.

7. The method of claim 6, wherein the class 2 CRISPR/Cas polypeptide is a Cpf1 polypeptide, a C2c1 polypeptide, a C2c3 polypeptide, or a C2c2 polypeptide.

8. The method of any one of claims 1-7, wherein modification of the genomic DNA is homozygous modification.

9. The method of any one of claims 1-7, wherein modification of the genomic DNA is heterozygous modification.

10. The method of any one of claims 1-7, wherein the modification comprises deletion of genomic DNA, insertion of a nucleic acid into the genomic DNA, or both deletion of genomic DNA and insertion of a nucleic acid into the genomic DNA.

11. The method of any one of claims 1-7, wherein the modification comprises inversion of genomic DNA.

12. The method of any one of claims 1-7, wherein the modification comprises insertion of a nucleic acid into genomic DNA.

13. The method of any one of claims 1-7, wherein the modification comprises replacement of genomic DNA.

14. The method of claim 12 or claim 13, comprising introducing into the zygote a donor DNA.

15. The method of any one of claims 1-14, wherein the zygote is a rodent zygote.

16. The method of claim 15, wherein the zygote is a mouse zygote or a rat zygote.

17. The method of any one of claims 1-14, wherein the zygote is a rabbit zygote, a cat zygote, a dog zygote, or a horse zygote.

18. The method of any one of claims 1-14, wherein the zygote is an ungulate zygote.

19. The method of any one of claims 1-14, wherein the zygote is a human zygote.

20. The method of any one of claims 1-14, wherein the zygote is a non-human primate zygote.

21. The method of any one of claims 1-20, wherein the electroporation comprises:
a) combining, in an electroporation container a zygote or a plurality of zygotes in a suitable liquid medium with an equal volume of an RNP complex, forming a zygote/RNP complex composition; and b) electroporating the zygote/RNP complex composition with one or more pulses at 30 V, wherein each of the one or more pulses is a 1-millisecond to 6 millisecond pulse.

22. The method of claim 21, wherein said electroporation comprises electroporating with 2 or more pulses at 30 V, wherein each of the 2 or more pulses is a 3-millisecond pulse.

23. The method of claim 21, wherein said electroporation comprises electroporating with 6 pulses of 30 V each, wherein each of the 6 pulses is a 3-millisecond pulse.

24. The method of any one of claims 1-23, wherein the RNP is present in the electroporation composition at a concentration of from 5 μM to 16 μM.

25. The method of any one of claims 1-23, wherein the RNP is present in the electroporation composition at a concentration of 8 μM.

26. The method of any one of claims 1-25, wherein at least 50%, at least 60%, at least 70%, at least 80%, at least 85%, at least 90%, at least 95%, or at least 98%, of the zygotes are viable after electroporation with the RNP.

27. The method of any one of claims 1-26, wherein the genomic modification occurs via homology-directed repair (HDR) or non-homologous end joining (NHEJ).

28. The method of any one of claims 1-27, wherein the genomic modification occurs via HDR, and wherein the efficiency of HDR is at least 50%, at least 60%, at least 70%, at least 80%, at least 90%, or 100%.

29. The method of any one of claims 1-27, wherein the genomic modification occurs via NHEJ, and wherein the efficiency of NHEJ is at least 50%, at least 60%, at least 70%, at least 80%, at least 90%, or 100%.

30. A method of modulating transcription in a mammalian zygote, the method comprising introducing into the zygote a ribonucleoprotein (RNP) comprising an enzymatically inactive CRISPR/Cas9 polypeptide complexed with a CRISPR/Cas guide RNA that hybridizes to a target sequence within the genomic DNA of the zygote, wherein said introducing is by electroporation of an electroporation composition comprising the RNP and the zygote, and wherein said introducing results in modulation of transcription of a gene comprising the target sequence.

31. The method of claim 30, wherein the zygote is a rodent zygote.

32. The method of claim 30, wherein the zygote is a mouse zygote or a rat zygote.

33. The method of claim 30, wherein the zygote is a rabbit zygote, a cat zygote, a dog zygote, or a horse zygote.

34. The method of claim 30, wherein the zygote is an ungulate zygote.

35. The method of claim 30, wherein the zygote is a human zygote.

36. The method of claim 30, wherein the zygote is a non-human primate zygote.

37. The method of any one of claims 30-36, wherein the electroporation comprises:
a) combining, in an electroporation container a zygote or a plurality of zygotes in a suitable liquid medium with an equal volume of an RNP complex, forming a zygote/RNP complex composition; and b) electroporating the zygote/RNP complex composition with one or more pulses at 30 V, wherein each of the one or more pulses is a 1-millisecond to 6 millisecond pulse.

38. The method of claim 37, wherein said electroporation comprises electroporating with 2 or more pulses at 30 V, wherein each of the 2 or more pulses is a 3-millisecond pulse.

39. The method of claim 37, wherein said electroporation comprises electroporating with 6 pulses of 30 V each, wherein each of the 6 pulses is a 3-millisecond pulse.

40. A method of labelling a genomic DNA in a mammalian zygote, the method comprising introducing into the zygote a ribonucleoprotein (RNP) comprising an enzymatically inactive CRISPR/Cas9 polypeptide complexed with a CRISPR/Cas guide RNA that hybridizes to a target sequence within the genomic DNA of the zygote, wherein said introducing is by electroporation of an electroporation composition comprising the RNP and the zygote, and wherein said introducing results in labelling of the genomic DNA.

41. The method of claim 40, wherein the zygote is a rodent zygote.

42. The method of claim 41, wherein the zygote is a mouse zygote or a rat zygote.

43. The method of claim 40, wherein the zygote is a rabbit zygote, a cat zygote, a dog zygote, or a horse zygote.

44. The method of claim 40, wherein the zygote is an ungulate zygote.

45. The method of claim 40, wherein the zygote is a human zygote.

46. The method of claim 40, wherein the zygote is a non-human primate zygote.

47. The method of any one of claims 40-46, wherein the electroporation comprises:
a) combining, in an electroporation container a zygote or a plurality of zygotes in a suitable liquid medium with an equal volume of an RNP complex, forming a zygote/RNP complex composition; and b) electroporating the zygote/RNP complex composition with one or more pulses at 30 V, wherein each of the one or more pulses is a 1-millisecond to 6 millisecond pulse.

48. The method of claim 47, wherein said electroporation comprises electroporating with 2 or more pulses at 30 V, wherein each of the 2 or more pulses is a 3-millisecond pulse.

49. The method of claim 47, wherein said electroporation comprises electroporating with 6 pulses of 30 V each, wherein each of the 6 pulses is a 3-millisecond pulse.

50. A method of delivering a ribonucleoprotein (RNP) complex into a mammalian zygote, the method comprising electroporating a composition comprising the mammalian zygote and the RNP complex, thereby delivering the RNP complex into the zygote.

51. The method of claim 50, wherein the RNP complex comprises an siRNA, an shRNA, a modified RNA, or a DNA nucleic acid.

52. A method of delivering a nucleic acid into a mammalian zygote, the method comprising electroporating a composition comprising the mammalian zygote and the nucleic acid, thereby delivering the nucleic acid into the zygote.

53. A method of delivering a polypeptide into a mammalian zygote, the method comprising electroporating a composition comprising the mammalian zygote and the polypeptide, thereby delivering the polypeptide into the zygote.

54. The method of any one of claims 50-53, wherein the zygote is a rodent zygote.

55. The method of claim 54, wherein the zygote is a mouse zygote or a rat zygote.

56. The method of any one of claims 50-53, wherein the zygote is a rabbit zygote, a cat zygote, a dog zygote, or a horse zygote.

57. The method of any one of claims 50-53, wherein the zygote is an ungulate zygote.

58. The method of any one of claims 50-53, wherein the zygote is a human zygote.

59. The method of any one of claims 50-53, wherein the zygote is a non-human primate zygote.

60. The method of any one of claims 50-53, wherein the electroporation comprises:
a) combining, in an electroporation container a zygote or a plurality of zygotes in a suitable liquid medium with an equal volume of the RNP complex, the nucleic acid, or the polypeptide, forming an electroporation composition; and b) electroporating the electroporation composition with one or more pulses at 30 V, wherein each of the one or more pulses is a 1-millisecond to 6 millisecond pulse.

61. The method of claim 60, wherein electroporation comprises electroporating with 2 or more pulses at 30 V, wherein each of the 2 or more pulses is a 3-millisecond pulse.

62. The method of claim 60, wherein electroporation comprises electroporating with 6 pulses of 30 V each, wherein each of the 6 pulses is a 3-millisecond pulse.
